(19) United States
(12) Patent Application Publication
(10) Pub. No.: US 2023/0263827 A1
(43) Pub. Date: Aug. 24, 2023

(54) EXPANDED MEMORY SUBSETS OF GAMMA DELTA T CELLS FOR IMMUNOTHERAPY

(71) Applicant: SERHAT GUMRUKCU, Los Angeles, CA (US)

(72) Inventor: Serhat GUMRUKCU, Los Angeles, CA (US)

(21) Appl. No.: 18/043,660

(22) PCT Filed: Sep. 2, 2021
(86) PCT No.: PCT/US2021/048897

§ 371 Date: Mar. 1, 2023

Related U.S. Application Data
(60) Provisional application No. 63/073,850, filed on Sep. 2, 2020.

(51) Int. Cl. A61K 35/17; C12N 5/0783; A61P 31/14; A61K 31/4965; A61K 31/706; A61K 31/4178; C07K 16/24

(52) U.S. Cl. CPC A61K 35/17; C12N 5/0636; A61P 31/14; A61K 31/4965; A61K 31/706; A61K 31/4178; C07K 16/248; C12N 2501/999

(57) ABSTRACT

The present disclosure provides compositions comprising enriched gamma delta T cells and methods for generating and expanding the enriched gamma delta T cells. In certain embodiments, these enriched gamma delta T cells are used for eliciting an immune response in a subject for methods of treating and/or preventing a coronavirus infection.

### CROSS-REFERENCE TO RELATED APPLICATIONS

This application claims the benefit of and priority to U.S. Provisional Application No. 63/073,850, filed Sep. 2, 2020, which is incorporated by reference in its entirety.

### SEQUENCE LISTING

The instant application contains a Sequence Listing with XX sequences, which has been submitted via EFS-Web and is hereby incorporated herein by reference in its entirety. Said ASCII copy, created on XXX XX, 2021, is named 46876WO_CRF_sequencelisting.txt, and is XX,XXX bytes in size.

### BACKGROUND

Globalization has an effect on both the transmission of pathogens and the availability of a pool of susceptible individuals. Global travel means that pathogens can travel around the world more easily. It also makes the pool of susceptible people larger. As a consequence, any new infectious disease, arising for example by mutation of an animal pathogen (e.g. MERS, SARS, bird flu, Ebola, HIV, etc.), has the opportunity to spread to susceptible individuals, and to be maintained within the human population for the long term.

The SARS-CoV-2 is a recently emerged highly pathogenic human coronavirus. This disease has been declared a pandemic by the World Health Organization (WHO) and is having severe effects on both individual lives and economies around the world. Infection with SARS-CoV-2 is characterized by a broad spectrum of clinical syndromes, which range from asymptomatic disease or mild influenza-like symptoms to severe pneumonia and acute respiratory distress syndrome

In view of the high morbidity and mortality that can follow infection with SARS-CoV-2, there is an urgent need for new treatments and vaccines for these deadly coronavirus infections.

### SUMMARY

Disclosed herein are compositions comprising gamma delta T cells which are enriched for Vgamma9 Vdelta2 (Vγ9Vδ2) T cells. In some embodiments, the composition comprises at least 80% Vgamma9 Vdelta2 (Vγ9Vδ2) T cells, and wherein the Vgamma9 Vdelta2 (Vγ9Vδ2) cells are specifically enriched for CD45RA⁻CD27⁻ effector memory (EM) and CD45RA⁻CD27⁺ central memory (CM) cells, when compared to a reference or control gamma delta T cell composition.

In some embodiments, the composition comprises at least 85% Vgamma9 Vdelta2 (Vγ9Vδ2) T cells. In some embodiments, the composition is capable of eliciting an immune response against a coronavirus when administered to a host. In some embodiments, the immune response comprises at least one of a humoral and cellular immune response.

Further provided herein is an enriched gamma delta T cell composition enriched for Vgamma9 Vdelta2 (Vγ9Vδ2) T cells produced by a method comprising:
a) isolating γδ T cells from a sample;
b) culturing the γδ T cells with at least one cytokine and a bisphosphonate or bisphosphonate derivative, in a suitable medium;
c) further incubating the γδ T cells with a peptide antigen that exhibits at least 70% homology to a coronavirus antigen; and
d) isolating and purifying the Vgamma9 Vdelta2 (Vγ9Vδ2) T cells to produce an enriched gamma delta T cell composition, wherein the Vgamma9 Vdelta2 (Vγ9Vδ2) T cells are specifically enriched for CD45RA⁻CD27⁻ effector memory (EM) and CD45RA⁻CD27⁺ central memory (CM) cells, when compared to a reference or control gamma delta T cell composition.

In some embodiments, the sample is whole blood or PBMCs.

In some embodiments, the peptide antigen has 80% homology to a coronavirus spike protein. In some embodiments, the spike protein sequence shares at least 90% homology with a coronavirus spike protein. In some embodiments, the spike protein sequence is SEQ ID NO: 1, or an optimized sequence thereof. In some embodiments, the spike protein sequence can comprise the entire sequence SEQ ID NO: 1 or amino acids 990-1749 comprising the ectodomain of SEQ ID NO: 1.

In certain embodiments, the purified γδT cells express at least two of the following markers: CD11 a, HLA-DR, CD86, CCR7, CXCR5, CD69, and are also CD45RA−. In certain embodiments, the purified γδT cells exhibit a decrease in two or more T cell exhaustion markers selected from group consisting of CTLA-4, LAG3, BTLA4, and TIM3.

Further provided herein is an allogeneic γδT cell composition produced by any of the methods described herein. In certain embodiments, the composition comprises at least about 10⁹ purified γδ T cells, wherein the γδ T cells comprise at least about 80% Vgamma9 Vdelta2 positive cells, and wherein the Vgamma9 Vdelta2 (Vγ9Vδ2) T cells are specifically enriched for CD45RA⁻CD27⁻ effector memory (EM) and CD45RA⁻CD27⁺ central memory (CM) cells, when compared to a reference or control gamma delta T cell composition.

Further provided herein is a pharmaceutical composition comprising the γδT cells of any of the enriched compositions described herein.

In certain embodiments, the pharmaceutical composition comprises, optionally, a pharmaceutically acceptable carrier, diluent, adjuvant and/or additive. In certain embodiments, the composition is capable of inducing an immunological response against coronavirus in a subject.

In certain embodiments, the method includes inducing a humoral and cellular response against coronavirus. In certain embodiments, the immune response is induced by a regimen comprising one or at least two administrations. In certain embodiments, the coronavirus is SARS-CoV-2 (COVID-19). In certain embodiments, the composition is for use in diminishing or preventing a coronavirus infection in a mammalian subject.

In certain embodiments, preventing comprises inducing coronavirus-specific immunity against SARS-CoV-2 (COVID-19).

Additionally, disclosed herein is a method of diminishing or preventing a coronavirus infection in a mammalian subject comprising administering an effective amount of the γδT cells or compositions described herein to the subject.

Additionally, disclosed herein is a method of inducing cellular and or humoral immunity in a mammalian subject, comprising administering an effective amount of the γδT cells or compositions described herein to the subject.

Additionally, disclosed herein is a method of eliciting an immune response in a subject, comprising administering an effective amount of the γδT cells or compositions described herein to the subject.

Additionally, disclosed herein is a method of inducing neutralizing antibodies against SARS-CoV-2 in a subject, comprising administering an effective amount of the γδT cells or compositions described herein to the subject.

In certain embodiments, the wherein the method includes inducing a humoral response against the coronavirus.

In certain embodiments, the humoral response is induced by a regimen comprising at least one or at least two administrations.

In some embodiments, the method further comprises administering an effective amount of at least one second therapeutic agent selected from the group consisting of: an antiviral agent, antibacterial agent, an angiotensin receptor blocker (ARB), an IL-6 inhibitor, hydroxychloroquine, chloroquine, an anticoagulant or and COVID-19 immune serum or plasma.

In some embodiments, the at least one second therapeutic agent is an antiviral agent.

In some embodiments, the antiviral agent is favipiravir.

In some embodiments, the second therapeutic agent is remdesivir.

In some embodiments, the at least one second therapeutic agent is an antibacterial agent.

In some embodiments, the antibacterial agent is selected from the group consisting of azithromycin, tobramycin, aztreonam, ciprofloxacin, meropenem, cefepime, cetadizine, imipenem, piperacillin-tazobactam, amikacin, gentamicin and levofloxacin.

In some embodiments, the antibacterial agent is azithromycin.

In some embodiments, the at least one second therapeutic agent is an ARB.

In some embodiments, the ARB is losartan.

In some embodiments, the ARB is valsartan.

In some embodiments, the at least one second therapeutic agent is an IL-6 inhibitor.

In some embodiments, the IL-6 inhibitor is selected from the group consisting of: an anti-IL-6 receptor antibody or an antigen binding fragment thereof, an anti-IL-6 antibody or an antigen binding fragment thereof, and a JAK/STAT inhibitor.

In some embodiments, the IL-6 inhibitor is an anti-IL-6 receptor antibody, or antigen binding fragment thereof.

In some embodiments, the anti-IL-6 receptor antibody is tocilizumab or sarilumab.

In some embodiments, the IL-6 inhibitor is an anti-IL-6 antibody, or antigen binding fragment thereof.

In some embodiments, the anti-IL-6 antibody is selected from the group consisting of ziltivekimab, siltuximab, gerilimzumab, sirukumab, clazakizumab, olokizumab, VX30 (VOP-R003; Vaccinex), EB-007 (EBI-029; Eleven Bio), and FM101 (Femta Pharmaceuticals, Lonza).

In some embodiments, the IL-6 inhibitor is a JAK/STAT inhibitor.

In some embodiments, the JAK/STAT inhibitor is selected from the group consisting of ruxolotinib, tofacitinib, and baricitinib.

In some embodiments, the anticoagulant is selected from the group consisting of enoxaparin, heparin, low-molecular weight heparin, dabigatran, rivaroxaban, apixaban, edoxaban, and fondaparinux.

In another aspect, provided herein is a kit comprising a container comprising the composition of enriched gamma delta T cells, and instructions for using the kit.

In some embodiments, the kit further comprises a separate container comprising normal saline.

In some embodiments, the kit further comprises materials suitable for intravenous administration and optionally additional materials for oral administration.

### BRIEF DESCRIPTION OF THE DRAWINGS

These and other features, aspects, and advantages of the present invention become better understood with regard to the following description, and accompanying drawings, where:

FIGS. 1A-1B are graphs that illustrate the increase in γδ T cells after COVID-19 infection, and the effect of expressing the SARS-CoV-2 spike protein on ex vivo expansion of γδ T cells.

FIGS. 2A-2C are graphs showing the effects of the SARS-CoV-2 spike protein expression on protein levels.

FIG. 3 is table showing phenotypic characteristics and expansion rates of pre-infection, seronegative and convalescent (post-infection) γδ T cells.

FIGS. 4A-4C shows the cytolytic and non-cytolytic effect of anti-SARs-CoV-2 activities on ex vivo expanded convalescent γδ T cells.

FIG. 5 is a table of COVID-19 biomarkers.

### DETAILED DESCRIPTION

The COVID-19 pandemic caused by SARS-CoV-2 has resulted in over 25 million cases with 844,000 deaths worldwide. Given the inconsistent and apparently short-lived anti-SARS-CoV-2 antibody profiles in COVID-19 convalescent individuals, the role of certain types of cellular immunity against the infection was analyzed. Analyses of the immunophenotyping and T cell repertoires from ten COVID-19 convalescent trial participants revealed that their γδ T cell populations were activated during the infection. In several subjects the comparison between pre- and post-infection immune cell profiles demonstrated that certain memory subsets of Vγ9Vδ2 T cell populations were selectively expanded, and these cells demonstrated potent antiviral activities in ex vivo experiments. In particular, the Vgamma9 Vdelta2 (Vγ9Vδ2) T cells are specifically enriched for CD45RA⁻CD27⁻ effector memory (EM) and CD45RA⁻CD27⁺ central memory (CM) cells, when compared to a baseline or control gamma delta T cell composition.

Moreover, ex vivo expanded γδ T cells from these individuals displayed potent antigen presenting cell (APC) functions, suggesting not only therapeutic, but also potential protective immunotherapy as a vaccine, as an allogeneic cellular vaccine.

γδ T cells, a subgroup of T cells based on the γδ TCR, when compared with conventional T cells (αβ T cells), make up a very small proportion of T cells. However, its various subgroups are widely distributed in different parts of the human body and are attractive effectors for infectious disease immunity. γδ T cells are activated and expanded by nonpeptidic antigens (P-Ags), major histocompatibility complex (MHC) molecules, and lipids which are associated with different kinds of pathogen infections. (Zhao et al. J. of Immunology Research, Volume 2018, Article ID 5081634, 15 pages).

Gamma delta T cells are not MHC restricted (Tanaka Y et al., 1995) and enriched gamma delta T cell compositions, as described herein can be produced by ex vivo expansion methods under certain conditions, and can act as a useful cell therapy as described herein for eliciting an immune response to SARS-CoV-2 and thus treating and/or preventing COVID-19.

Coronaviridae is a family of viruses (e.g., MERS-CoV and Severe Acute Respiratory Syndrome (SARS-CoV)) that primarily infect the upper respiratory and gastrointestinal tracts of mammals and birds, and that are responsible for acute and chronic diseases of the respiratory, hepatic, gastrointestinal and neurological systems. Coronaviruses are large, enveloped, positive-stranded RNA viruses. They have the largest genome among all RNA viruses, typically ranging from 27 to 32 kb. The genome is packed inside a helical capsid formed by the nucleocapsid protein (N) and further surrounded by an envelope. Associated with the viral envelope are at least three structural proteins: The membrane protein (M) and the envelope protein (E) are involved in virus assembly, whereas the spike protein (S) mediates virus entry into host cells.

The spike protein is the viral membrane protein that is responsible for cell entry and includes an S1 domain, which is responsible for binding the cell surface receptor, and an S2 domain, which is a membrane-anchored subunit. Some coronaviruses also encode an envelope-associated hemagglutinin-esterase protein (HE). Among these structural proteins, the spike forms large protrusions from the virus surface, giving coronaviruses the appearance of having crowns (hence their name; corona in Latin means crown). In addition to mediating virus entry, the spike is an important determinant of viral host range and tissue tropism and a major inducer of host immune responses.

The virions of each coronavirus are approximately 100 nm with a crown-like appearance because of the club-shaped spike (S) proteins projecting from the surface of the envelope. The spike protein is the viral membrane protein that is responsible for cell entry and includes an S1 domain, which is responsible for binding the cell surface receptor, and an S2 domain, which is a membrane-anchored subunit.

Upon entering an infected cell, coronaviruses transcribe their RNA and the viruses replicate in the cytoplasm of the infected cell. Replication is mediated by the synthesis of an antisense RNA strand, which is provided as a template for additional viral genomes and transcription. The viruses then assemble and are released from the infected cell.

Infected patients may present with any of the following: fever, high temperature (>37.3° C.), cough, myalgia, sputum production, headache, haemoptysis, diarrhoea, dyspnoea and in some cases, acute respiratory distress syndrome (ARDS), acute cardiac injury or secondary infection.

Enriched gamma delta T cell compositions as described herein are expected to shorten or prevent these symptoms as well as the length and duration of any infection caused by SARS-CoV-2, particularly in patients who are only recently exposed to the virus, i.e. those who are not severely ill. Additionally, the enriched gamma delta T cell compositions as described herein are also expected to be effective as a vaccine to prevent COVID-19, or at the least, to prevent several symptoms.

1.1. Definitions

Unless defined otherwise, all technical and scientific terms used herein have the meaning commonly understood by a person skilled in the art to which this invention belongs.

As used herein, the following terms have the meanings ascribed to them below.

coroAs used herein, the terms “patient” and “subject” are used interchangeably, and may be taken to mean any living organism which may be treated with compounds of the present invention. As such, the terms “patient” and “subject” include, but are not limited to, humans, veterinary animals (e.g., cats, dogs, cows, horses, sheep, pigs, etc.) and experimental animal models of diseases (e.g., mice, rats, ferrets, monkeys, etc.). In a preferred embodiment, the subject is a human.

As used herein “preventing” a disease refers to inhibiting the full development of a disease.

The term “biological sample” refers to any tissue, cell, fluid, or other material derived from an organism (e.g., human subject). In certain embodiments, the biological sample is serum or blood.

The terms “treat,” “treated,” “treating”, or “treatment” as used herein have the meanings commonly understood in the medical arts, and therefore do not require cure or complete remission, and therefore include any beneficial or desired clinical results.

“Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). Improvements in any conditions can be readily assessed according to standard methods and techniques known in the art. The population of subjects treated by the method of the disease includes subjects suffering from the undesirable condition or disease, as well as subjects at risk for development of the condition or disease.

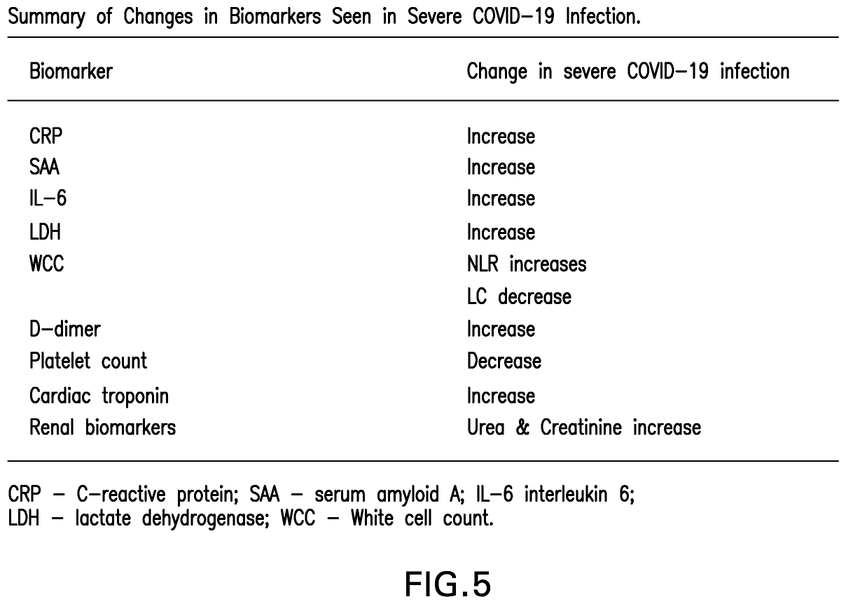

Nonlimiting examples of such beneficial or desired clinical results are prolonging survival as compared to expected survival if not receiving treatment, an improvement in any of the COVID-associated biomarkers (e.g., any of those in FIG. 5) reduced probability of requiring intubation and mechanical ventilation, reduced number of days on mechanical ventilation, reduced days in the ICU, reduced days of total hospitalization.

The term “therapeutically effective amount” is an amount that is effective to ameliorate a symptom of a disease. A “therapeutically effective amount” of a composition is an amount sufficient to achieve a desired therapeutic effect, and therefore does not require cure or complete remission A therapeutically effective amount can be a “prophylactically effective amount” as prophylaxis can be considered therapy.

The term “ameliorating” refers to any therapeutically beneficial result in the treatment of a disease state, e.g., a neurodegenerative disease state, including prophylaxis, lessening in the severity or progression, remission, or cure thereof.

As used herein, the term “neutralizing antibody” refers to an antibody that binds to a pathogen (e.g., a virus) and interferes with its ability to infect a cell. Non-limiting examples of neutralizing antibodies include antibodies that bind to a viral particle and inhibit successful transduction, e.g., one or more steps selected from binding, entry, trafficking to the nucleus, and transcription of the viral genome. Some neutralizing antibodies may block a virus at the post-entry step.

The term “immune response” refers to a response of a cell of the immune system (e.g., a B-cell, T-cell, macrophage or polymorphonucleocyte) to a stimulus such as an antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). Active immune response can be contrasted with passive immunity, which can be acquired through the transfer of substances such as, e.g., an antibody, transfer factor, thymic graft, and/or cytokines from an actively immunized host to a non-immune host.

As used herein in connection with a viral infection and vaccination, the terms “protective immune response” or “protective immunity” refer to an immune response that that confers some benefit to the subject in that it prevents or reduces the infection or prevents or reduces the development of a disease associated with the infection. As an example, the presence of SARS-CoV-2 neutralizing antibodies in a subject can indicate the presence of a protective immune response in the subject.

As used herein, “interleukin 6 (IL-6)” or “IL-6 polypeptide” refers to a human polypeptide or fragment thereof having at least about 85% or greater amino acid identity to the amino acid sequence provided at NCBI Accession No. NP_000591 and having IL-6 biological activity. IL-6 is a pleotropic cytokine with multiple biologic functions. Exemplary IL-6 biological activities include immunostimulatory and pro-inflammatory activities.

Unless otherwise specified, “IL-6 antagonist” is used synonymously with “IL-6 inhibitor” and refers to an agent that is capable of decreasing the biological activity of IL-6. IL-6 antagonists include agents that decrease the level of IL-6 polypeptide in serum, including agents that decrease the expression of an IL-6 polypeptide or nucleic acid; agents that decrease the ability of IL-6 to bind to the IL-6R; agents that decrease the expression of the IL-6R; and agents that decrease signal transduction by the IL-6R receptor when bound by IL-6. In preferred embodiments, the IL-6 antagonist decreases IL-6 biological activity by at least about 10%, 20%, 30%, 50%, 70%, 80%, 90%, 95%, or even 100%. As further described below, IL-6 antagonists include IL-6 binding polypeptides, such as anti-IL-6 antibodies and antigen binding fragments or derivatives thereof, IL-6R binding polypeptides, such as anti-IL-6R antibodies and antigen binding fragments or derivatives thereof, and synthetic chemical molecules, such as JAK1 and JAK3 inhibitors.

The term “IL-6 antibody” or “anti-IL-6 antibody” refers to an antibody that specifically binds IL-6 ligand. Anti-IL-6 antibodies include monoclonal and polyclonal antibodies that are specific for IL-6 ligand, and antigen-binding fragments or derivatives thereof. IL-6 antibodies are described in greater detail below.

The term “C-reactive protein” or “CRP” refers to a polypeptide or fragment thereof having at least about 85% or greater amino acid identity to the amino acid sequence provided at NCBI Accession No. NP_000558 and having complement activating activity. CRP levels increase in response to inflammation and can be measured with an hsCRP (high-sensitivity C-reactive protein) test.

The term “biological sample” refers to any tissue, cell, fluid, or other material derived from an organism (e.g., human subject). In certain embodiments, the biological sample is serum or blood.

As used herein, “pre-treatment” means prior to the first administration of enriched gamma delta T cell compositions according to the methods described herein. Pre-treatment does not exclude, and often includes the prior administration of treatments other than enriched gamma delta T cell compositions according to the methods described herein.

As used herein, “post-treatment” means after the administration of enriched gamma delta T cell compositions according to the methods described herein. Post-treatment includes after any administration of enriched gamma delta T cell compositions according to the methods described herein, at any dosage described herein.

Any of the biological indicators listed in FIG. 5 can be utilized as biomarkers indicating a patient in need for treatment with enriched gamma delta T cell compositions. Additionally, any one of combination of these biomarkers can be utilized to show an improvement in patient outcome by exhibiting an improved level following treatment with the enriched gamma delta T cell compositions described herein.

“Allogeneic” as used herein means cells from different individuals of the same species.

Severe acute respiratory syndrome beta coronavirus 2 and SARS-CoV-2 (COVID-19) are used interchangeably herein. In a specific embodiment, the virus is SARS-CoV-2, also referred to as nCoV-2, nCoV2 or 2019-nCoV. The terms “nCoV2”, “nCoV-2”, “SARS-CoV-2” and “SARS-CoV-2 (COVID-19)” are used interchangeably herein. In particular embodiments, the patient has severe acute respiratory syndrome (SARS). In particular embodiments, the patient has coronavirus disease 2019 (COVID-19).

The amino acid sequence of the full length COVID-19 spike glycoprotein (S-protein) can be found at: Genbank ID 43740568.

SEQ ID NO: 1
MFVFLVLLPLVSSQCVNLTTRTQLPPAYTNSFTRG
VYYPDKVFRSSVLHSTQDLFLPFFSNVTWFHAIH
VSGTNGTKRFDNPVLPFNDGVYFASTEKSNIIRGW
IFGTTLDSKTQSLLIVNNATNVVIKVCEFQFCNDP
FLGVYYHKNNKSWMESEFRVYSSANNCTFEYVSQP
FLMDLEGKQGNFKNLREFVFKNIDGYFKIYSKHTP
INLVRDLPQGFSALEPLVDLPIGINITRFQTLLAL
HRSYLTPGDSSSGWTAGAAAYYVGYLQPRTFLLKY
NENGTITDAVDCALDPLSETKCTLKSFTVEKGIYQ
TSNFRVQPTESIVRFPNITNLCPFGEVFNATRFAS
VYAWNRKRISNCVADYSVLYNSASFSTFKCYGVSP
TKLNDLCFTNVYADSFVIRGDEVRQIAPGQTGKIA
DYNYKLPDDFTGCVIAWNSNNLDSKVGGNYNYLYR
LFRKSNLKPFERDISTEIYQAGSTPCNGVEGFNCY
FPLQSYGFQPTNGVGYQPYRVVVLSFELLHAPATV
CGPKKSTNLVKNKCVNFNFNGLTGTGVLTESNKKF
LPFQQFGRDIADTTDAVRDPQTLEILDITPCSFGG
VSVITPGTNTSNQVAVLYQDVNCTEVPVAIHADQL
TPTWRVYSTGSNVFQTRAGCLIGAEHVNNSYECDI
PIGAGICASYQTQTNSPRRARSVASQSIIAYTMSL
GAENSVAYSNNSIAIPTNFTISVTTEILPVSMTKT
SVDCTMYICGDSTECSNLLLQYGSFCTQLNRALTG
IAVEQDKNTQEVFAQVKQIYKTPPIKDFGGFNFSQ
ILPDPSKPSKRSFIEDLLFNKVTLADAGFIKQYGD
CLGDIAARDLICAQKFNGLTVLPPLLTDEMIAQYT
SALLAGTITSGWTFGAGAALQIPFAMQMAYRFNGI
GVTQNVLYENQKLIANQFNSAIGKIQDSLSSTASA
LGKLQDVVNQNAQALNTLVKQLSSNFGAISSVLND
ILSRLDKVEAEVQIDRLITGRLQSLQTYVTQQLIR
AAEIRASANLAATKMSECVLGQSKRVDFCGKGYHL
MSFPQSAPHGVVFLHVTYVPAQEKNFTTAPAICHD
GKAHFPREGVFVSNGTHWFVTQRNFYEPQUITTDN
TFVSGNCDVVIGIVNNTVYDPLQPELDSFKEELDK
YFKNHTSPDVDLGDISGINASVVNIQKEIDRLNEV
AKNLNESLIDLQELGKYEQYIKWPWYIWLGFIAGL
IAIVMVTIMLCCMTSCCSCLKGCCSCGSCCKFDED
DSEPVLKGVKLHYT

The nucleotide sequence of the full length Covid-19 spike glycoprotein (S-protein) can be found at: Genbank ID NC_045512 REGION: 21563 . . . 0.25384.

Any one or combination of the immunogenic antigens from SARS-CoV-2 can be utilized as peptide antigens for ex-vivo methods of producing the enriched gamma delta T cells described herein.

The coronavirus has immunogenic antigens/polypeptides including at least: envelope proteins, membrane proteins, spike proteins, and hemagglutinin. The immunogenic antigen can be a sequence that is at least 70% homologous or exhibits at least 70% identity to any of these polypeptides, fragments thereof, or epitopes from a coronavirus. Most preferably, the virus is SARS-CoV-2.

Multiple variants of SARS-CoV-2 have arisen throughout the world in the eighteen months since the virus was first detected. Three mutations in particular, B.1.1.7, B.1.351, and P.1, have become dominant and have increased the effectiveness of the virus. The N501Y mutation, which is in the receptor-binding-domain of the spike protein. The 501Y.V2 and P.1 variants both have two additional receptor-binding-domain mutations, K417N/T and E484K. These mutations increase the binding affinity of the receptor-binding domain to the angiotensin-converting enzyme 2 (ACE2) receptor. Four key concerns stemming from the emergence of the new variants are their effects on viral transmissibility, disease severity, reinfection rates (i.e., escape from natural immunity), and vaccine effectiveness (i.e., escape from vaccine-induced immunity).

The enriched gamma delta T cell compositions described herein can be produced from exposure to any one or combination of variants of the SARS-CoV-2 spike protein (for example, one based on reference protein found at Genbank: NC_045512), or any desired antigenic protein (Nature Reviews Microbiology Vol. 19, pgs. 409-424 (2021). see also the database: Global Initiative on Sharing All Influenza Data (GISAID), or in combination with another antigenic peptide, according to the methods described herein.

1.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Treatment with the enriched gamma delta T cell compositions described herein will diminish or alleviate any one or more of the symptoms or complications associated with COVID-19 infection, as described below.

In certain embodiments, prophylactic treatment with the enriched gamma delta T cell compositions described herein will prevent a full-blown COVID-19 infection, or in certain embodiments, the cell treatment will elicit an immune response which serves to diminish certain COVID-19 symptoms, a selection of which are listed below, such that the patient/host is essentially asymptomatic.

### 2. COVID Symptoms and Related Features

2.1. Fever

In some embodiments, the patient has fever. In some embodiments, the patient has a body temperature greater than 37.5° C. In some embodiments, the body temperature is 37.6° C. or greater, 37.7° C. or greater, 37.8° C. or greater, 37.9° C. or greater, 38° C. or greater, 38.1° C. or greater, 38.2° C. or greater, 38.3° C. or greater, 38.4° C. or greater, 38.5° C. or greater, 38.6° C. or greater, 38.7° C. or greater, 38.8° C. or greater, 38.9° C. or greater, 39° C. or greater, 39.1° C. or greater, 39.2° C. or greater, 39.3° C. or greater, 39.4° C. or greater, 39.5° C. or greater, 39.6° C. or greater, 39.7° C. or greater, 39.8° C. or greater, 39.9° C. or greater, 40° C. or greater, 40.1° C. or greater, 40.2° C. or greater, 40.3° C. or greater, 40.4° C. or greater, 40.5° C. or greater, 40.6° C. or greater, 40.7° C. or greater, 40.8° C. or greater, 40.9° C. or greater, 41° C. or greater, or 42° C. or greater. In some embodiments, the patient has a body temperature greater than 37.5° C. for 24 hours or more, 48 hours or more, 72 hours or more, 96 hours or more, 5 days or more, 6 days or more, 1 week or more, 1.5 weeks or more, or 2 weeks or more. In typical embodiments, the body temperature is measured from clinically accessible measurement sites on the patient. In various embodiments, the measurement site is the patient's forehead, temple, and/or other external body surfaces. In some embodiments, the measurement site is the oral cavity, rectal cavity, axilla area, or tympanic membrane.

2.2. Reduced Blood Oxygen Saturation

In some embodiments, the patient has a blood oxygen saturation level (SpO₂) of less than 95%. In some embodiments, the patient has a blood oxygen saturation level (SpO₂) of less than 94%. In some embodiments, the patient has a blood oxygen saturation level (SpO₂) of 93% or less. In some embodiments, the patient has an SpO₂ level of 92% or less, 91% or less, 90% or less, 85% or less, 80% or less, 75% or less, 70% or less, 65% or less, 60% or less, 55% or less, 50% or less, 45% or less, 40% or less, 35% or less, 30% or less, or 25% or less. In some embodiments, the patient requires mechanical ventilation and/or supplemental oxygen.

2.3. Pneumonia

In some embodiments, the patient has pneumonia.

2.4. Hospitalization

In some embodiments, the patient is hospitalized.

2.5. Patient Age

In some embodiments, the patient is older than 60 years old. In some embodiments, the patient is older than 50 years old. In some embodiments, the patient is older than 50, 51, 52, 53, 54, 55, 56, 57, 58, 59, or 60 years old. In some embodiments, the patient is younger than 60, 59, 58, 57, 56, 55, 54, 53, 52, 51, or 50 years old. In some embodiments the patient is a young adult between the age of 20-35. In some embodiments, the patient is middle aged, between the age of 35-50. In some embodiments, the patient is a teenager between the age of 13-19. In some embodiments, the patient is a child between the age of 5-12. In alternative embodiments, the patient is a toddler between the age of 1-4. In further embodiments, the patient is an infant between the age of newborn to one year old.

2.6. Additional Agents

In some embodiments, the methods of the present disclosure further comprise administering an effective amount of at least one second therapeutic agent.

In certain embodiments, one or more corticosteroids may be administered to the patient, either prior to, concurrently, or post-administration of enriched gamma delta T cell compositions.

In certain embodiments, the second therapeutic agent is selected from the group consisting of an antiviral agent, an antibacterial agent, an anticoagulant, an angiotensin receptor blocker (ARB), an IL-6 inhibitor, hydroxychloroquine, chloroquine, and COVID-19 immune serum or plasma.

In certain embodiments, anticoagulants are administered to the patient in addition to enriched gamma delta T cell compositions. In particular embodiments, low molecular weight heparin (LMWH) is administered to the patient undergoing enriched gamma delta T cell compositions treatment. In certain embodiments, unfractionated heparin is administered to the patient undergoing enriched gamma delta T cell compositions treatment. In certain embodiments, patients are provided DVT prophylaxis under certain conditions.

2.6.1. Anti-Viral Agents

In some embodiments, the method of the present disclosure further comprises administering an effective amount of an anti-viral agent.

In some embodiments, the anti-viral agent is selected from the group consisting of: favipiravir, remdesivir, and a combination of lopinavir and ritonavir.

In particular embodiments, the anti-viral agent is favipiravir.

In particular embodiments, the anti-viral agent is remdesivir.

In particular embodiments, the anti-viral agent is a combination of lopinavir and ritonavir.

2.6.2. Antibacterial Agents

In some embodiments, the method of the present disclosure further comprises administering an antibacterial agent. In some embodiments, the antibacterial agent is selected from the group consisting of azithromycin, tobramycin, aztreonam, ciprofloxacin, meropenem, cefepime, cetadizine, imipenem, piperacillin-tazobactam, amikacin, gentamicin and levofloxacin. In certain embodiments, the antibacterial agent is azithromycin.

2.7. Anticoagulants

In some embodiments, the methods herein further comprise administering an anticoagulant. In certain embodiments, the anticoagulant can be selected from the group of anticoagulants comprising enoxaparin, heparin, low-molecular weight heparin, dabigatran, rivaroxaban, apixaban, edoxaban, and fondaparinux.

In one embodiment, the anticoagulant is an FXa targeting coagulant. In one embodiment, the anticoagulant is heparin (anti-thrombin cofactor), warfarin (Coumadin or vitamin K antagonist), dabigatran (Pradaxa, direct thrombin inhibitor), enoxaparin (Lovenox), edoxaban (Savaysa), rivaroxaban and/or apixaban (two direct FXa inhibitors).

In additional embodiments, the anticoagulant can be any of the following: rivaroxaban (Xarelto), apixaban (Eliquis), Dalteparin, fragmin, otamixaban, fidexaban, razaxaban, fondaparinux (Arixtra), idraparinux, DU-176b, PMD-3112, YM-150, KFA-1982, EMD-503982, MCM-17, MLN-1021, DX 9065a, DPC 906, JTV 803, PRT054021, PRT064445, SSR-126512 or SSR-128428.

2.8. Angiotensin Receptor Blocker (ARB)

In some embodiments, the methods herein further comprise administering an ARB.

In particular embodiments, the ARB is selected from losartan, valsartan, azilsartan, candesartan, eprosartan, irgesartan, olmesartan, and telmisartan.

2.9. IL-6 Antagonists

In certain embodiments, the patient is further administered an IL-6 antagonist. In some embodiments, the IL-6 inhibitor or antagonist is selected from the group consisting of: an anti-IL-6 receptor antibody or an antigen binding fragment thereof, an anti-IL-6 antibody or an antigen binding fragment thereof; and a JAK/STAT inhibitor.

2.10. Anti-IL-6 Receptor Antibodies

In various embodiments, the IL-6 antagonist is an anti-IL-6 receptor (anti-IL-6R) antibody or antigen-binding fragment or derivative thereof.

In typical embodiments, the anti-IL-6R reduces the biological activity of IL-6 receptor.

In some embodiments, the IL-6 antagonist is an anti-IL-6R monoclonal antibody. In some embodiments, the IL-6 antagonist is a polyclonal composition comprising a plurality of species of anti-IL-6R antibodies, each of the plurality having unique CDRs.

In some embodiments, the anti-IL-6R antibody is a Fab, Fab′, F(ab′)2, Fv, scFv, (scFv)2, single chain antibody molecule, dual variable domain antibody, single variable domain antibody, linear antibody, or V domain antibody.

In some embodiments, the anti-IL-6R antibody comprises a scaffold. In certain embodiments, the scaffold is Fe, optionally human Fc. In some embodiments, the anti-IL-6R antibody comprises a heavy chain constant region of a class selected from IgG, IgA, IgD, IgE, and IgM. In certain embodiments, the anti-IL-6R antibody comprises a heavy chain constant region of the class IgG and a subclass selected from IgG1, IgG2, IgG3, and IgG4.

In some embodiments, the IL-6 antagonist is immunoconjugate or fusion protein comprising an IL-6R antigen-binding fragment.

In some embodiments, the antibody is bispecific or multispecific, with at least one of the antigen-binding portions having specificity for IL-6 receptor.

In some embodiments, the antibody is fully human. In some embodiments, the antibody is humanized. In some embodiments, the antibody is chimeric and has non-human V regions and human C region domains. In some embodiments, the antibody is murine.

In typical embodiments, the anti-IL-6R antibody has a KD for binding human IL-6 receptor of less than 100 nM. In some embodiments, the anti-IL-6R antibody has a KD for binding human IL-6 receptor of less than 75 nM, 50 nM, 25 nM, 20 nM, 15 nM, or 10 nM. In particular embodiments, the anti-IL-6R antibody has a KD for binding human IL-6 receptor of less than 5 nM, 4 nM, 3 nM, or 2 nM. In selected embodiments, the anti-IL-6R antibody has a KD for binding human IL-6 receptor of less than 1 nM, 750 pM, or 500 pM. In specific embodiments, the anti-IL-6R antibody has a KD for binding human IL-6 receptor of no more than 500 pM, 400 pM, 300 pM, 200 pM, or 100 pM.

In typical embodiments, the anti-IL-6R antibody has an elimination half-life following intravenous administration of at least 7 days. In certain embodiments, the anti-IL-6R antibody has an elimination half-life of at least 14 days, at least 21 days, or at least 30 days.

In some embodiments, the anti-IL-6R antibody has a human IgG constant region with at least one amino acid substitution that extends serum half-life as compared to the unsubstituted human IgG constant domain.

2.11. Tocilizumab and Derivatives

In certain embodiments, the anti-IL-6R antibody or antigen-binding portion thereof comprises all six CDRs of tocilizumab. In particular embodiments, the antibody or antigen-binding portion thereof comprises the tocilizumab heavy chain V region and light chain V region. In specific embodiments, the antibody is the full-length tocilizumab antibody.

In various embodiments, the anti-IL-6R antibody is a derivative of tocilizumab.

In some embodiments, the tocilizumab derivative includes one or more amino acid substitutions in the tocilizumab heavy and/or light chain V regions.

In certain embodiments, the tocilizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of the tocilizumab anti-IL-6R antibody, while retaining specificity for human IL-6 receptor.

In certain embodiments, the tocilizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of tocilizumab. The percent sequence identity is determined using BLAST algorithms using default parameters.

In certain embodiments, the tocilizumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of tocilizumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL 6 receptor).

2.12. Sarilumab and Derivatives

In certain embodiments, the anti-IL-6R antibody or antigen-binding portion thereof comprises all six CDRs of sarilumab. In particular embodiments, the antibody or antigen-binding portion thereof comprises the sarilumab heavy chain V region and light chain V region. In specific embodiments, the antibody is the full-length sarilumab antibody.

In various embodiments, the anti-IL-6R antibody is a derivative of sarilumab.

In some embodiments, the sarilumab derivative includes one or more amino acid substitutions in the sarilumab heavy and/or light chain V regions.

In certain embodiments, the sarilumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of the sarilumab anti-IL-6R antibody, while retaining specificity for human IL-6 receptor.

In certain embodiments, the sarilumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of sarilumab. The percent sequence identity is determined using BLAST algorithms using default parameters.

In certain embodiments, the sarilumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of sarilumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL 6 receptor).

2.13. Vobarilizumab and Derivatives

In certain embodiments, the anti-IL-6R antibody or antigen-binding portion thereof comprises all six CDRs of vobarilizumab. In particular embodiments, the antibody or antigen-binding portion thereof comprises the vobarilizumab heavy chain V region and light chain V region. In specific embodiments, the antibody is the full-length vobarilizumab antibody.

In various embodiments, the anti-IL-6R antibody is a derivative of vobarilizumab.

In some embodiments, the vobarilizumab derivative includes one or more amino acid substitutions in the vobarilizumab heavy and/or light chain V regions.

In certain embodiments, the vobarilizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of the vobarilizumab anti-IL-6R antibody, while retaining specificity for human IL-6 receptor.

In certain embodiments, the vobarilizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of vobarilizumab. The percent sequence identity is determined using BLAST algorithms using default parameters.

In certain embodiments, the vobarilizumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of vobarilizumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL 6 receptor).

2.14. Other Anti-IL-6R Antibodies and Derivatives

In certain embodiments, the anti-IL-6R antibody or antigen-binding portion thereof comprises all six CDRs of an antibody selected from the group consisting of: SA237 (Roche), NI-1201 (NovImmune), and an antibody described in US 2012/0225060. In particular embodiments, the antibody or antigen-binding portion thereof comprises the heavy chain V region and light chain V region of an antibody selected from the group consisting of: SA237 (Roche), NI-1201 (NovImmune), and an antibody described in US 2012/0225060. In specific embodiments, the antibody is a full-length selected from the group consisting of: SA237 (Roche), NI-1201 (NovImmune), and an antibody described in US 2012/0225060.

In various embodiments, the anti-IL-6R antibody is a derivative of an antibody selected from the group consisting of: SA237 (Roche), NI-1201 (NovImmune), or an antibody described in US 2012/0225060.

2.15. Anti-IL-6:IL-6R Complex Antibodies

In various embodiments, the IL-6 antagonist is an antibody specific for the complex of IL-6 and IL-6R. In certain embodiments, the antibody has the six CDRs of an antibody selected from those described in US 2011/0002936, which is incorporated herein by reference in its entirety.

2.16. Anti-IL-6 Antibodies

In various embodiments, the IL-6 antagonist is an anti-IL-6 antibody or antigen-binding fragment thereof.

In typical embodiments, the anti-IL-6 antibody or antigen-binding fragment thereof neutralizes the biological activity of human IL-6. In some embodiments, the neutralizing antibody prevents binding of IL-6 to the IL-6 receptor. In certain embodiments, the neutralizing antibody prevents binding of IL-6 to the soluble IL-6 receptor. In certain embodiments, the neutralizing antibody prevents binding of IL-6 to the membrane-bound IL-6 receptor. In certain embodiments, the neutralizing antibody prevents binding of IL-6 to both the soluble IL-6 receptor and the membrane-bound IL-6 receptor.

In some embodiments, the IL-6 antagonist is an anti-IL-6 monoclonal antibody. In some embodiments, the IL-6 antagonist is a polyclonal composition comprising a plurality of species of anti-IL-6 antibodies, each of the plurality having unique CDRs.

In some embodiments, the anti-IL-6 antibody is selected from the group consisting of: ziltivekimab, siltuximab, gerilimzumab, sirukumab, clazakizumab, olokizumab, VX30 (VOP-R003; Vaccinex), EB-007 (EBI-029; Eleven Bio), and FM101 (Femta Pharmaceuticals, Lonza). In some embodiments, the antigen-binding fragment is a fragment of an antibody selected from the group consisting of: ziltivekimab, siltuximab, gerilimzumab, sirukumab, clazakizumab, olokizumab, VX30 (VOP-R003; Vaccinex), EB-007 (EBI-029; Eleven Bio), and FM101 (Femta Pharmaceuticals, Lonza).

2.17. IL-6 Antagonist Peptides

In various embodiments, the IL-6 antagonist is an antagonist peptide.

In certain embodiments, the IL-6 antagonist is C326 (an IL-6 inhibitor by Avidia, also known as AMG220), or FE301, a recombinant protein inhibitor of IL-6 (Ferring International Center S.A., Conaris Research Institute AG). In some embodiments, the anti-IL-6 antagonist comprises soluble gp130, FE301 (Conaris/Ferring).

2.18. JAK and STAT Inhibitors

In various embodiments, the IL-6 antagonist is an inhibitor of the JAK signaling pathway. In some embodiments, the JAK inhibitor is a JAK1-specific inhibitor. In some embodiments, the JAK inhibitor is a JAK3-specific inhibitor. In some embodiments, the JAK inhibitor is a pan-JAK inhibitor. In certain embodiments, the JAK inhibitor is selected from the group consisting of tofacitinib (Xeljanz), decernotinib, ruxolitinib, upadacitinib, baricitinib, filgotinib, lestaurtinib, pacritinib, peficitinib, momelotinib, INCB-039110, ABT-494, INCB-047986 and AC-410.

In various embodiments, the IL-6 antagonist is a STAT3 inhibitor. In a specific embodiment, the inhibitor is AZD9150 (AstraZeneca, Isis Pharmaceuticals), a STAT3 antisense molecule.

In typical embodiments, small molecule JAK inhibitors and STAT inhibitors are administered orally.

In various embodiments, the inhibitor is administered once or twice a day at an oral dose of 0.1-1 mg, 1-10 mg, 10-20 mg, 20-30 mg, 30-40 mg, or 40-50 mg. In some embodiments, the inhibitor is administered once or twice a day at a dose of 50-60 mg, 60-70 mg, 70-80 mg, 80-90 mg, or 90-100 mg. In some embodiments, the inhibitor is administered at a dose of 0.1, 0.5, 1, 2, 5, 10, 15, 20, 25, 30, 35, 40, 45, or 50 mg PO once or twice a day. In some embodiments, the inhibitor is administered at a dose of 75 mg or 100 mg PO once or twice a day.

2.19. Hydroxychloroquine and Chloroquine

In some embodiments, the method further comprises administering an anti-malarial agent. In certain embodiments, the anti-malarial agent is hydroxychloroquine. In certain embodiments, the anti-malarial agent is chloroquine.

2.20. COVID-19 Immune Serum or Plasma

In some embodiments, the method further comprises administering a COVID-19 immune serum or plasma, or a composition comprising isolated or recombinantly expressed anti-SARS-CoV-2 antibodies having sequences derived from COVID-19 immune serum or plasma.

2.21. Post-Treatment Reduction of IL-6 and C-Reactive Protein (CRP)

In some embodiments, the administration of an effective amount of enriched gamma delta T cell compositions reduces the patient's free serum IL-6 levels below pre-treatment levels. In various embodiments, the dosage regimen is adjusted to achieve a reduction in the patient's free serum IL-6 levels below pre-treatment levels.

In some embodiments, the free serum IL-6 level is decreased by at least 10% as compared to pre-treatment levels. In various embodiments, the free serum IL-6 level is decreased by at least 20%, 30%, 40%, 50%, 60%, 70%, 80%, or 90% as compared to pre-treatment levels. In certain embodiments, the free serum IL-6 level is decreased by at least 20% as compared to pre-treatment levels. In certain embodiments, the free serum IL-6 level is decreased by at least 30% as compared to pre-treatment levels. In certain embodiments, the free serum IL-6 level is decreased by at least 40% as compared to pre-treatment levels. In certain embodiments, the free serum IL-6 level is decreased by at least 50% as compared to pre-treatment levels. In certain embodiments, the free serum IL-6 level is decreased by at least 60% as compared to pre-treatment levels. In certain embodiments, the free serum IL-6 level is decreased by at least 70% as compared to pre-treatment levels. In certain embodiments, the free serum IL-6 level is decreased by at least 80% as compared to pre-treatment levels. In certain embodiments, the free serum IL-6 level is decreased by at least 90% as compared to pre-treatment levels.

In some embodiments, the administration of an effective amount of enriched gamma delta T cell compositions, reduces the patient's serum CRP levels below pre-treatment levels. In various embodiments, the dosage regimen is adjusted to achieve a reduction in the patient's serum CRP levels below pre-treatment levels.

In some embodiments, the post-treatment CRP level is no more than 45 mg/L. In certain embodiments, the post-treatment CRP level is no more than 40 mg/L. In certain embodiments, the post-treatment CRP level is no more than 30 mg/L. In certain embodiments, the post-treatment CRP level is no more than 20 mg/L. In certain embodiments, the post-treatment CRP level is no more than 10 mg/L. In certain embodiments, the post-treatment CRP level is no more than 5 mg/L. In certain embodiments, the post-treatment CRP level is no more than 2.5 mg/L. In certain embodiments, the post-treatment CRP level is no more than 2 mg/L. In certain embodiments, the post-treatment CRP level is no more than 1 mg/L.

In some embodiments, the CRP level is decreased by at least 10% as compared to pre-treatment levels. In various embodiments, the CRP level is decreased by at least 20%, 30%, 40%, 50%, 60%, 70%, 80%, or 90% as compared to pre-treatment levels. In certain embodiments, the CRP level is decreased by at least 20% as compared to pre-treatment levels. In certain embodiments, the CRP level is decreased by at least 30% as compared to pre-treatment levels. In certain embodiments, the CRP level is decreased by at least 40% as compared to pre-treatment levels. In certain embodiments, the CRP level is decreased by at least 50% as compared to pre-treatment levels. In certain embodiments, the CRP level is decreased by at least 60% as compared to pre-treatment levels. In certain embodiments, the CRP level is decreased by at least 70% as compared to pre-treatment levels. In certain embodiments, the CRP level is decreased by at least 80% as compared to pre-treatment levels. In certain embodiments, the CRP level is decreased by at least 90% as compared to pre-treatment levels.

2.22. Other Post-Treatment Endpoints

In some embodiments, administering an effective amount of enriched gamma delta T cell compositions to the patient prevents a hyperinflammatory response in the patient. In some embodiments, the dosage regimen is adjusted to prevent a hyperinflammatory response in the patient.

In some embodiments, administering an effective amount of enriched gamma delta T cell compositions to the patient results in a reduction in body temperature. In some embodiments, the patient, post-treatment with an effective amount of enriched gamma delta T cell compositions, has a body temperature of 37.5° C. or below. In some embodiments, the patient, post-treatment with an effective amount of enriched gamma delta T cell compositions, has a body temperature ranging from of 36 to 37.5° C.

In some embodiments, administering an effective amount of enriched gamma delta T cell compositions to the patient results in a reduction in the risk of respiratory morbidity and mortality. In some embodiments, the dose is adjusted to reduce the risk of respiratory morbidity and mortality.

In some embodiments, administering an effective amount of enriched gamma delta T cell compositions to the patient results in a reduction in the patient's need for supplemental oxygen. In some embodiments, the dose is adjusted to reduce the patient's need for supplemental oxygen.

In some embodiments, administering an effective amount of enriched gamma delta T cell compositions to the patient results in eliminating the patient's need for assisted ventilation. In some embodiments, the dose is adjusted to eliminate the patient's need for assisted ventilation.

Additionally, any of the primary and/or secondary endpoints can be met by administering an effective amount of enriched gamma delta T cell compositions as described herein.

2.23. Kits

Additionally, certain components or embodiments of the compositions can be provided in a kit. For example, the enriched gamma delta T cell composition, as well as the related buffers or other components related to administration can be provided in separate containers and packaged as a kit, alone or along with separate containers of any of the other agents from any pre-conditioning or post-conditioning steps, and optional instructions for use. In some embodiments, the kit may comprise ampoules, disposable syringes, capsules, vials, tubes, or the like. In some embodiments, the kit may comprise a single dose container or multiple dose containers comprising the embodiments herein. In some embodiments, each dose container may contain one or more unit doses. In some embodiments, the kit may include an applicator.

In some embodiments, the kits include all components needed for the various stages of treatment. In some embodiments, the compositions may have preservatives or be preservative-free (for example, in a single-use container). In some embodiments, the kit may comprise materials for intravenous administration. In some embodiments, the kit may comprise an additional component in a separate container.

2.24. Methods of Producing and Expanding Gamma Delta T cells to Make Enriched Compositions for Therapeutic Treatment or Vaccination for SARS-CoV-2

Methods for obtaining the enriched gamma delta T cells described herein may include the isolation of peripheral blood mononuclear cells (PBMCs) from blood or leukapheresis material using density gradient centrifugation. Isolated PBMCs may be cryopreserved prior to expansion in culture. In certain embodiments freshly isolated PBMCs (or those resuscitated from cryopreservation) are inoculated into growth media containing human recombinant IL-2 (e.g. at a concentration of up to 1000 U/ml) and Zoledronic acid (e.g. 5 pM, or any suitable bisphosphonate or bisphosphonate derivative). The enriched gamma delta T lymphocyte population may be activated and selectively proliferated from the PBMCs via the addition of zoledronic acid (day 0) and the continuous inclusion of IL-2 over a 14 day culture period, as well as incubation with the SARS-CoV-2 spike protein (at any suitable concentration, which is typically 100 nM), or ectodomain, or any combination of SARS-CoV-2 spike protein fragments or other SARS-CoV-2 antigenic peptides.

In certain embodiments, the cell suspension may be serially expanded (typically at a 1:2 split ratio) over this time period. In certain embodiments, fourteen days after culture initiation the cells can be harvested and resuspended in lactated ringers solution and HSA prior to transfer to an infusion bottle containing 100 ml saline solution. In certain embodiments, the expansion protocol may also include the combinatorial usage of zoledronic acid, IL-2, and a feeder cell population, such as K562. In some embodiments, the expansion protocol may also include the combinatorial usage of zoledronic acid, IL-2, a feeder cell population, such as K562, and an anti-CD3 antibody, such as OKT3. (See, Tan W K, Tay J C K, Zeng J, Zheng M, Wang S. J Immunol Sci. (2018); 2(3): 6-12.)

Following expansion, in certain embodiments, the enriched gamma delta T cell compositions exhibit the following minimum specifications; greater than 80% of total cells are T lymphocytes (CD3 positive), gamma delta T lymphocytes comprise 60% or greater of the total T lymphocyte population (Vgamma9 positive), NK cells are less than 15% of the total T lymphocyte population (CD3 negative/CD56 positive), Cytotoxic T cells are below 10% of total T lymphocyte population (CD3/CD8 positive) and T helper cells are below 5% of total T lymphocyte population (CD3/CD4 positive). In additional embodiments, the Vgamma9 Vdelta2 (Vγ9Vδ2) are specifically enriched for CD45RA⁻CD27⁻ effector memory (EM) and CD45RA⁻CD27⁺ central memory (CM) cells, when compared to a reference or control gamma delta T cell composition.

In certain embodiments, enriched gamma delta T cell compositions meeting these specifications can be used as the starting material for the generation of high purity allogeneic cell banks which will aim to have greater than 99% gamma delta T cells.

In some embodiments the enriched gamma delta T cell composition comprises at least 70% of the Vγ9Vδ2 subset. In some embodiments the gamma delta T cell composition comprises at least 71% of the Vγ9Vδ2 subset. In some embodiments the gamma delta T cell composition comprises at least 72% of the Vγ9Vδ2 subset. In some embodiments the gamma delta T cell composition comprises at least 73% of the Vγ9Vδ2 subset. In some embodiments the gamma delta T cell composition comprises at least 74% of the Vγ9Vδ2 subset. In some embodiments the gamma delta T cell composition comprises at least 75% of the Vγ9Vδ2 subset. In some embodiments the gamma delta T cell composition comprises at least 76% of the Vγ9Vδ2 subset. In some embodiments the gamma delta T cell composition comprises at least 77% of the Vγ9Vδ2 subset. In some embodiments the gamma delta T cell composition comprises at least 78% of the Vγ9Vδ2 subset. In some embodiments the gamma delta T cell composition comprises at least 79% of the Vγ9Vδ2 subset. In some embodiments the gamma delta T cell composition comprises at least 80% of the Vγ9Vδ2 subset. In some embodiments the gamma delta T cell composition comprises between 80-85% of the Vγ9Vδ2 subset. In some embodiments the gamma delta T cell composition comprises between 82-87% of the Vγ9Vδ2 subset. In some embodiments the gamma delta T cell composition comprises between 85-90% of the Vγ9Vδ2 subset. In some embodiments the gamma delta T cell composition comprises between 87-92% of the Vγ9Vδ2 subset. In some embodiments the gamma delta T cell composition comprises between 90-95% of the Vγ9Vδ2 subset. In some embodiments the gamma delta T cell composition comprises between 92-97% of the Vγ9Vδ2 subset. In some embodiments the gamma delta T cell composition comprises at least 97% of the Vγ9Vδ2 subset. In some embodiments the gamma delta T cell composition comprises at least 98% of the Vγ9Vδ2 subset. In some embodiments the gamma delta T cell composition comprises at least 99% of the Vγ9Vδ2 subset. In some embodiments the gamma delta T cell composition comprises 100% of the Vγ9Vδ2 subset.

Preferably, the enriched gamma delta T cells produced by certain methods disclosed herein do not produce, or do not produce high levels of, IL-17 and/or IL-10. Preferably, they do not actively support tumor or T regulatory (Treg) cell growth. In some cases, a low or very low proportion of gamma delta T cells in the population of cells produced by the method produce IL17 and/or IL10. Preferably, fewer than 5% of the cells in the population, fewer than 4% of the cells in the population, fewer than 3% of the cells in the population, fewer than 2% of the cells in the population, or fewer than 1% of the cells in the population produce IL-17 and/or IL-10.

Methods disclosed herein may be used to generate enriched gamma delta T cells useful for antigen presentation, and/or producing proinflammatory cytokines. Also disclosed are enriched gamma delta T cells produced by these methods.

Enriched gamma delta T cells disclosed herein may highly express antigen presentation markers, cell costimulation markers and/or effector markers. In this context “highly expressed” means at a level equal to, or preferably higher than, a gamma delta T cell generated in the presence of IL-2 alone. “IL-2 alone” refers to culture when IL-2 is the only cytokine that has been added to the culture, or the only interleukin added to the culture. Certain gamma delta T cells disclosed herein express markers at 1.1, 1.2, 1.3, 1.4 or 1.5 times more than the expression of the same marker in a gamma delta T cell generated in the presence of IL-2 alone. Certain enriched gamma delta T cells disclosed herein express markers at 2, 2.5, 3, or 3.5 times more than the expression of the same marker in a gamma delta T cell cultured in the presence of IL-2 alone.

Expression of markers may be determined by any suitable means. Expression may be gene expression or protein expression. Gene expression can be determined e.g. by detection of mRNA encoding the marker, for example by quantitative real-time PCR (qRT-PCR). Protein expression can be determined e.g. by detection of the marker, for example by antibody-based methods, for example by western blot, immunohistochemistry, immunocytochemistry, flow cytometry, or ELISA.

In preferred embodiments “expression” refers to protein expression of the relevant marker at/on the cell surface, and can be detected by flow cytometry using an appropriate marker-binding molecule.

Certain enriched gamma delta T cells disclosed herein highly express one or more antigen presentation markers, such as HLA-ABC, and/or HLA-DR. Certain enriched gamma delta T cells disclosed herein highly express one or more cell costimulation markers, such as CD80, CD83, CD86, CD40 and/or ICAM-1.

These markers may be associated with presenting antigens to, and activating, CD4⁺ and CD8⁺ T cells.

Certain enriched gamma delta T cells disclosed herein highly express one or more effector markers, such as CCR5, CCR6, CCR7, CD69, CD27 and/or NKG2D. These markers may be associated with homing of gamma delta T cells to lymph nodes, and interaction of gamma delta T cells with CD4⁺ and CD8⁺ T cells.

Certain enriched gamma delta T cells disclosed herein express higher levels of ICAM-1 than a gamma delta T cell generated in the presence of IL-2 alone. Such enriched gamma delta T cells may have been generated in the presence of IL-2 and another interleukin, such as IL-7, IL-15, IL-18, IL-21, or both IL-18 and IL-21. Such gamma delta T cells may be particularly useful where antigen presentation activity may be desirable.

Certain enriched gamma delta T cells disclosed herein express higher levels of CD83 and/or CD80 than a gamma delta T cell generated in the presence of IL-2 alone. Such enriched gamma delta T cells may have been generated in the presence of IL-2 and another interleukin, such as IL-7, IL-15 or IL-18. Such gamma delta T cells may be particularly useful where antigen presentation activity may be desirable.

Certain enriched gamma delta T cells disclosed herein express higher levels of CCR5, CCR7, CD69, CD27 and/or NKG2D than a gamma delta T cell generated in the presence of IL2. Such enriched gamma delta T cells may be particularly useful where effector activity may be desirable. Enriched gamma delta T cells disclosed herein may express at least 1.5, at least 2, at least 2.5 or at least 3 times more CCR5 than a gamma delta T cell generated in the presence of IL-2 alone.

2.25. Antigen Presentation Phenotypes

The enriched gamma delta T cells may exhibit antigen presentation phenotypes. That is, gamma delta T cells may capture antigens and enable their recognition by other T cells, such as CD4⁺ and CD8⁺ T cells, including al T cells, thereby activating those T cells. Gamma delta T cells generated/expanded according to the methods of the present invention may be employed as antigen-presenting cells in methods for expanding T cells having a desired specificity, e.g. virus-specific T cells.

Accordingly, the present invention provides a method for generating/expanding a population of antigen-specific T cells, comprising stimulating T cells by culture in the presence of gamma delta T cells generated/expanded according to the present invention, by further incubating the culture of T cells with a peptide of a coronavirus.

As used herein a “peptide” refers to a chain of two or more amino acid monomers linked by peptide bonds, which is 50 amino acids or fewer in length.

The antigen may be a peptide or polypeptide antigen. In some embodiments the antigen is associated with an infectious disease, such as a coronavirus. In some embodiments, the antigen is expressed by, or expression is upregulated in, a cell infected with an infectious agent (e.g. a virus or intracellular pathogen). In some embodiments, the antigen is an antigen of an infectious agent (e.g. peptide/polypeptide of an infectious agent).

In connection with various aspects of the present invention, a cell (e.g. a gamma delta T cell) may present a peptide of an antigen as a consequence of infection by an infectious agent comprising/encoding the antigen/fragment thereof, uptake by the cell of the antigen/fragment thereof or expression of the antigen/fragment thereof. The presentation is typically in the context of an MHC molecule at the cell surface of the antigen-presenting cell.

It will be appreciated that reference to “a peptide” herein encompasses plural peptides. For example, cells presenting a peptide of an antigen may present plural peptides of the antigen. Methods for generating and/or expanding populations of e.g. antigen-specific T cells typically include several rounds of stimulation of T cells with antigen presenting cells presenting peptide of the antigen of interest (i.e. the virus for which the T cells are specific).

In one aspect, the present invention provides a method for generating or expanding a population of T cells specific for a virus, comprising stimulating T cells (e.g. within a population of immune cells, e.g. PBMCs, PBLs) by culture in the presence of enriched gamma delta T cells expanded according to the methods described herein by incubation with a coronavirus spike protein, ectodomain, combination thereof, or any fragment of a coronavirus antigen.

In aspects of the present invention wherein the gamma delta T cells generated/expanded according to the methods of the present invention are employed in methods to expand antigen-specific T cells, the gamma delta T cells may be treated in order that they express present one or more peptides of the relevant antigen. For example, the gamma delta T cells may be incubated or pulsed with peptides of the antigen according to methods well known to the skilled person. In certain embodiments, antigenic peptides may be provided in a library of peptide mixtures (corresponding to one or more antigens), which may be referred to as pepmixes. Peptides of pepmixes may e.g. be overlapping peptides of 8-10 amino acids in length, and may cover all or part of the amino acid sequence of the relevant antigen(s).

Activation of CD4⁺ and CD8⁺ T cells involves IFNγ and TNFα. Enriched gamma delta T cells produced by some of certain methods disclosed herein produce IFNγ and TNFα and may therefore be useful for antigen presentation, and activation of CD4⁺ and/or CD4⁺ T cells.

In some cases, the population of cells generated by certain methods disclosed herein comprises at least 45%, at least 50%, at least 60%, or at least 65% cells that produce at least one of IFNγ and TNFα. Preferably, the population of cells generated by certain methods disclosed herein comprises at least 20%, at least 25%, at least 30%, at least 35%, at least 40%, at least 45%, or at least 50% gamma delta T cells that produce both IFNγ. and TNFα.

Production of a given factor (e.g. IFNγ and TNFα) by gamma delta T cells can be measured by detecting gene or protein expression. Protein expression can be measured by various means known to those skilled in the art such as antibody-based methods, for example by ELISA, ELISPOT, western blot, immunohistochemistry, immunocytochemistry, flow cytometry or reporter-based methods. Production can also be determined by measuring levels of mRNA by quantitative real-time PCR (qRT-PCR), or by reporter-based methods.

In some embodiments, enriched gamma delta T cells generated/expanded by the methods disclosed herein display increased expression of one or more factors as compared to the level of expression by gamma delta T cells generated/expanded by traditional tumor cells e.g. a cancer cell or C666-1, Hep3B, DLD-1 or K562 cells). In some embodiments a factor may be selected from granzyme A, granzyme B, granulysin, perforin, IFNγ, IL-17A, IL-8, Eotaxin, IP-10, MIG, GRO A, MIUP-3A, I-TAC, MCP-1, RANTES, MIP-1A, MIP-1B and ENA-78.

2.26. Interleukins

Methods disclosed herein relate to the culture of PBMCs in the presence of one or more interleukins. Certain methods may involve culture in the presence of exogenous interleukin. That is, interleukin that has been added to the culture, such as added to the culture media. The interleukins employed in the methods of the present invention may be recombinantly produced, and/or obtained from a suitable source for clinical application.

In accordance with various aspects disclosed herein, where culture is performed in the “presence of” a given cytokine, the relevant cytokine (e.g. recombinant and/or exogenous cytokine) may have been added to the culture. Where culture is performed in the “absence of” a given cytokine, the relevant cytokine (e.g. recombinant and/or exogenous cytokine) will not have been added to the culture.

In some cases, the cells are cultured in media that has been supplemented with the one or more interleukins. In others, the media comprises the one or more interleukins. Some of certain methods involve culturing PBMCs in the presence of two or more interleukins simultaneously. That is, the culture comprises a plurality of interleukins, rather than sequential culture of the cells in each different cytokine individually. However, having cultured the cells in one particular interleukin, or combination of interleukins, the cells may be subsequently transferred to a further culture using a different interleukin or combination of interleukins.

IL-2 has been validated for use in generating gamma delta T cells for the clinic. In some methods disclosed herein, gamma delta T cells may be generated in the presence of at least 150 IU/ml, at least 160 IU/ml, at least 170 IU/ml, at least 180 IU/ml, at least 190 IU/ml, at least 200 IU/ml of IL2. Preferably, the gamma delta T cells are generated in the presence of 200 IU/ml IL-2.

In some embodiments IL-2 is added to the culture at a final concentration 50-500 IU/ml, 50-400 IU/ml, 50-300 IU/ml, 50-250 IU/ml, 50-200 IU/ml, 75-500 IU/ml, 75-400 IU/ml, 75-300 IU/ml, 75-250 IU/ml, 75-200 IU/ml, 100-500 IU/ml, 100-400 IU/ml, 100-300 IU/ml, 100-250 IU/ml, 100-200 IU/ml, 125-500 IU/ml, 125-400 IU/ml, 125-300 IU/ml, 125-250 IU/ml, 125-200 IU/ml, 150-500 IU/ml, 150-400 IU/ml, 150-300 IU/ml, 150-250 IU/ml, or 150-200 IU/ml.

As used herein, IU means International Unit, and is a measure of activity determined by an International Standard. The International Standard for 112 is NIBSC 86/504. In some methods disclosed herein, IL-2 may be used in combination with other cytokines. In particular, IL-2 may be used in combination with IL-15 or IL-21.

In some methods disclosed herein, gamma delta T cells may be generated in the presence of Interleukin 15 (IL-15) at a concentration of at least 2 ng/ml, at least 3 ng/ml, at least 4 ng/ml, at least 5 ng/ml, at least 6 ng/ml, at least 7 ng/ml, at least 8 ng/ml, at least 9 ng/ml or at least 10 ng/ml. Preferably, certain methods disclosed herein involve culture of gamma delta T cells in the presence of 10 ng/ml of IL-15.

In some embodiments IL-15 is added to the culture at a final concentration 1-30 ng/ml, 1-25 ng/ml, 1-20 ng/ml, 1-15 ng/ml, 1-10 ng/ml, 2-30 ng/ml, 2-25 ng/ml, 2-20 ng/ml, 2-15 ng/ml, 2-10 ng/ml, 3-30 ng/ml, 3-25 ng/ml, 3-20 ng/ml, 3-15 ng/ml, 3-10 ng/ml, 4-30 ng/ml, 4-25 ng/ml, 4-20 ng/ml, 4-15 ng/ml, 4-10 ng/ml, 5-30 ng/ml, 5-25 ng/ml, 5-20 ng/ml, 5-15 ng/ml, or 5-10 ng/ml. In some methods disclosed herein, IL-15 may be used alone or in combination with other cytokines. For example, IL-15 may be used in combination with IL-2, or IL-21 and IL-18.

In some methods disclosed herein, gamma delta T cells may be generated in the presence of Interleukin 21 (IL-21) at a concentration of at least 15 ng/ml, at least 20 ng/ml, at least 25 ng/ml, at least 5 ng/ml, at least 26 ng/ml, at least 27 ng/ml, at least 28 ng/ml, at least 29 ng/ml or at least 30 ng/ml. Preferably, certain methods disclosed herein involve culture of gamma delta T cells in the presence of 30 ng/ml of IL-21.

In some embodiments IL-21 is added to the culture at a final concentration 5-80 ng/ml, 5-70 ng/ml, 5-60 ng/ml, 5-50 ng/ml, 5-40 ng/ml, 5-30 ng/ml, 10-80 ng/ml, 10-70 ng/ml, 10-60 ng/ml, 10-50 ng/ml, 10-40 ng/ml, 10-30 ng/ml, 15-80 ng/ml, 15-70 ng/ml, 15-60 ng/ml, 15-50 ng/ml, 15-40 ng/ml, 15-30 ng/ml, 20-80 ng/ml, 20-70 ng/ml, 20-60 ng/ml, 20-50 ng/ml, 20-40 ng/ml, 20-30 ng/ml, 25-80 ng/ml, 25-70 ng/ml, 25-60 ng/ml, 25-50 ng/ml, 25-40 ng/ml, or 25-30 ng/ml.

In some methods disclosed herein, IL-21 may be used alone or in combination with other cytokines. For example, IL-21 may be used in combination with IL-2 or IL15. IL-21 may be used in combination with IL-18, and IL-2 or IL15.

In some methods disclosed herein, gamma delta T cells are generated in the presence of Interleukin 7 (IL-7) at a concentration of at least 2 ng/ml, at least 3 ng/ml, at least 4 ng/ml, at least 5 ng/ml, at least 6 ng/ml, at least 7 ng/ml, at least 8 ng/ml, at least 9 ng/ml or at least 10 ng/ml. Preferably, certain methods disclosed herein involve culture of gamma delta T cells in the presence of 10 ng/ml of IL-7.

In some embodiments IL-7 is added to the culture at a final concentration 1-30 ng/ml, 1-25 ng/ml, 1-20 ng/ml, 1-15 ng/ml, 1-10 ng/ml, 2-30 ng/ml, 2-25 ng/ml, 2-20 ng/ml, 2-15 ng/ml, 2-10 ng/ml, 3-30 ng/ml, 3-25 ng/ml, 3-20 ng/ml, 3-15 ng/ml, 3-10 ng/ml, 4-30 ng/ml, 4-25 ng/ml, 4-20 ng/ml, 4-15 ng/ml, 4-10 ng/ml, 5-30 ng/ml, 5-25 ng/ml, 5-20 ng/ml, 5-15 ng/ml, or 5-10 ng/ml.

In some methods disclosed herein, gamma delta T cells may be generated in the presence of Interleukin 18 (IL-18) at a concentration of at least 2 ng/ml, at least 3 ng/ml, at least 4 ng/ml, at least 5 ng/ml, at least 6 ng/ml, at least 7 ng/ml, at least 8 ng/ml, at least 9 ng/ml or at least 10 ng/ml. Preferably, certain methods disclosed herein involve culture of gamma delta T cells in the presence of 10 ng/ml of IL18.

In some embodiments IL-18 is added to the culture at a final concentration 1-30 ng/ml, 1-25 ng/ml, 1-20 ng/ml, 1-15 ng/ml, 1-10 ng/ml, 2-30 ng/ml, 2-25 ng/ml, 2-20 ng/ml, 2-15 ng/ml, 2-10 ng/ml, 3-30 ng/ml, 3-25 ng/ml, 3-20 ng/ml, 3-15 ng/ml, 3-10 ng/ml, 4-30 ng/ml, 4-25 ng/ml, 4-20 ng/ml, 4-15 ng/ml, 4-10 ng/ml, 5-30 ng/ml, 5-25 ng/ml, 5-20 ng/ml, 5-15 ng/ml, or 5-10 ng/ml.

Methods disclosed herein relate to the culture of gamma delta T cells in the presence of one or more Interleukin. In particular, methods disclosed herein relate to culture of gamma delta T cells in the presence of: IL-2 and IL-21; IL-15; IL-21; IL-15 and IL-21; IL-2 and IL-18; IL-15, IL-18 and IL-21; IL-2 and IL-7; IL-2 and IL-15; IL-2, IL-18 and IL-21; IL-15 and IL-7; or IL-15 and IL-18.

Certain methods disclosed herein relate to culture of gamma delta T cells in the presence of IL-15. In particular, methods disclosed herein relate to the culture of gamma delta T cells in the presence of IL-15 and IL-21. In some cases, the gamma delta T cells are generated in the presence of IL-15 and IL-21 and IL-18.

Certain methods disclosed herein relate to culture of gamma delta T cells in the presence of IL-21. In particular, methods disclosed herein relate to the culture of gamma delta T cells in the presence of IL-21 and IL-2, or IL-21 and IL-15. In some cases, the gamma delta T cells are generated in the presence of IL-21 and IL-2 and IL-18. In some cases, the gamma delta T cells are generated in the presence of IL21 and IL-15 and IL-18.

In the methods of the present disclosure, the one or more interleukins are added to the culture on one or more of days 1, 2, 3, 4, 5, 6, 7, 8, 9 and 10. In some embodiments the interleukins are added to the culture at the same time as, or after, the addition of an agent capable of stimulating the proliferation of gamma delta T cells (e.g. zoledronic acid). In some embodiments the interleukins are added on day 1 of the culture. In some embodiments the interleukins are added to the culture on day 3 of the culture. In some embodiments the interleukins are added to the culture on days 1 and 3 of the culture. In some embodiments the interleukins are added to the culture: daily, every 2 days, every 3 days, every 4 days or every 5 days.

In some embodiments the agent capable of stimulating the proliferation of gamma delta T cells is added at the same time as adding one or more interleukins to the culture.

2.27. T Cell Medium

Methods disclosed herein relate to the culture of gamma delta T cells in cell culture medium, and particularly in T cell medium. T cell medium is a liquid containing nutrients that supports the growth of T cells, such as amino acids, inorganic salts, vitamins, and sugars. As used here, the term T cell medium refers to medium that does not contain cytokines, such that the amount of cytokine in the culture may be manipulated through the addition of one or more cytokines. In some cases, the T cell medium does not contain interleukins, such that the amount of interleukin in the culture may be manipulated through the addition of one or more interleukins.

Suitable T cell medium includes Click's medium, or OpTimizer.RTM. (CTS.RTM.), medium. Stemline.RTM. T cell expansion medium (Sigma-Aldrich), AIM V.RTM. medium (CTS.RTM.), TexMACS.RTM. medium (Miltenyi Biotech), ImmunoCult.RTM. medium (Stem Cell Technologies), PRIME-XV.RTM. T-Cell Expansion XSFM (Irvine Scientific), Iscoves medium and RPMI-1640 medium.

In particular, certain methods disclosed herein relate to the culture of gamma delta T cells in Clicks medium, or OpTimizer.RTM. medium.

In certain aspects, certain methods disclosed herein relate to culture in OpTimizer.RTM. T cell medium (CTS.RTM.).

Medium used in the present invention may be serum free medium, or may comprise serum. In some methods, serum may be added to serum free medium.

In some embodiments the medium may comprise one or more cell culture medium additives. Cell culture medium additives are well known to the skilled person, and include antibiotics (e.g. penicillin, streptomycin), serum, L-glutamine, growth factors, etc.

2.28. Serum

Culture medium is commonly supplemented with serum in cell culture methods. Serum may provide factors required for cell attachment, grown and proliferation, and thus may act as a growth supplement.

Serum may be serum of human or animal origin. The serum may be human serum. Serum may be pooled human AB serum, FBS (Fetal Bovine Serum) or defined FBS. The serum may be autologous serum.

Preferably, the serum is a clinically acceptable serum. The serum may be sterile filtered. The serum may be heat-inactivated.

Some methods disclosed herein relate to the culture of gamma delta T cells in culture medium supplemented with 1%, 2%, 3%, 4%, 5%, 6%, 7%, 8%, 9%, 10%, 11%, 12%, 13%, 14% or 15% serum. In some cases, the culture medium may be supplemented with at least 1% serum, at least 2% serum, at least 3% serum, at least 4% serum, at least 5% serum, at least 6% serum, at least 7% serum, at least 8% serum, at least 9% serum, at least 10% serum, at least 11% serum, at least 12% serum, at least 13% serum, at least 14% serum, at least 15% serum.

In some methods, the culture medium may be supplemented with 10% serum, or at least 10% serum. In some cases, the culture medium may be supplemented with less than 30% serum, less than 25% serum, less than 20% serum, or less than 15% serum. In some cases, the culture medium may be supplemented with one of 1-20%, 1-15% or 1-10% serum. In some cases, the culture medium may be supplemented with one of 1-10%, 1-8% or 1-5% serum.

2.29. Compositions

The invention described herein also provides compositions comprising enriched gamma delta T cells produced according the methods described herein.

The enriched gamma delta T cells may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion.

Suitable formulations may comprise the enriched gamma delta T cells in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In particular embodiments, the compositions may be formulated for intramuscular administration.

In accordance with the compositions and treatment methods described herein are also provided for the production of pharmaceutically useful compositions, such methods of production may comprise one or more steps selected from: isolating/purifying gamma delta T cells produced according to the methods described herein; and/or mixing gamma delta T cells produced according to the methods described herein with a pharmaceutically acceptable carrier, adjuvant, excipient or diluent.

For example, a further aspect the invention described herein relates to a method of formulating or producing a medicament or pharmaceutical composition, comprising formulating a pharmaceutical composition or medicament by mixing enriched gamma delta T cells produced according to the methods described herein with a pharmaceutically acceptable carrier, adjuvant, excipient or diluent.

2.30. Pharmaceutical Compositions and Methods of Treatment

The enriched gamma delta T cells and pharmaceutical compositions according to the present invention find use in therapeutic and prophylactic methods.

The present invention provides an enriched gamma delta T cell or pharmaceutical composition according to the present invention for use in a method of medical treatment or prophylaxis.

The present invention also provides the use of an enriched gamma delta T cell or pharmaceutical composition according to the present invention in the manufacture of a medicament for treating or preventing a disease or disorder, namely COVID-19.

The present invention also provides a method of treating or preventing a disease or disorder, comprising administering to a subject a therapeutically or prophylactically effective amount of a gamma delta T cell or pharmaceutical composition according to the present invention.

The disease or disorder to be treated/prevented may be any disease/disorder which would derive therapeutic or prophylactic benefit from an increase in the number of gamma delta T cells.

Also as described herein, the methods of the present invention are useful for generating/expanding gamma delta T cells which are in turn useful as antigen presenting cells for use in methods for expanding antigen-specific T cells, e.g. virus-specific T cells useful in methods for treating/preventing diseases/disorders (e.g. viral disease).

In particular embodiments, the enriched compositions described herein are capable of eliciting both cellular and humoral immune responses against the SARS-CoV-2 virus.

2.31. Administration

Administration of an enriched gamma delta T cell or pharmaceutical composition according to the invention is preferably in a “therapeutically effective” or “prophylactically effective” amount, this being sufficient to show benefit to the subject.

The actual amount administered, and rate and time-course of administration, will depend on the nature and severity of the disease or disorder. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Multiple doses of enriched gamma delta T cells or composition may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

In some embodiments the enriched gamma delta T cells or pharmaceutical compositions of the present invention may be administered alone or in combination with one or more other agents, either simultaneously or sequentially dependent upon the condition to be treated/prevented.

In some embodiments enriched gamma delta T cells or pharmaceutical compositions disclosed herein may be administered in combination with an agent capable of activating gamma delta T cells e.g. an agent comprising a phospho antigen and/or aminobisphosphonate. In some embodiments the agent may be pamidronate or zoledronic acid.

Simultaneous administration refers to administration of the enriched gamma delta T cells/pharmaceutical composition and agent together, for example as a pharmaceutical composition containing both of (i) the enriched gamma delta T cells/pharmaceutical composition and (ii) the agent, in combined preparation or immediately after each other and optionally via the same route of administration, e.g. to the same artery, vein or other blood vessel.

Sequential administration refers to administration of one or other of the (i) enriched gamma delta T cells/pharmaceutical composition and (ii) the agent after a given time interval by separate administration. It is not required that the two agents are administered by the same route, although this is the case in some embodiments. The time interval may be any time interval.

### EXAMPLES

### Example 1: Immunophenotyping and T Cell Repertoires from Convalescent (Post-Infection) PBMCs

Analyses of the immunophenotyping and T cell repertoires from ten COVID-19 convalescent trial participants revealed that their γδ T cell populations were activated during the COVID-19 infection. In several subjects, the comparison between pre- and post-infection immune cell profiles demonstrated that certain memory subsets of Vγ9Vδ2 T cell populations were selectively expanded, and these cells demonstrated potent antiviral activities in ex vivo experiments. Surprisingly, ex vivo expanded γδ T cells from these individuals displayed potent antigen presenting cell (APC) functions, suggesting not only therapeutic, but also potential protective immunotherapy implications of these cells.

### Methods

Plasma and PBMC samples from COVID-19 seven convalescent individuals were collected under IRCM-2020-241 IRB approval. Three healthy PBMC donors (who had been routine healthy PBMC donors) were recovering from COVID-19 and were also recruited to the study to compare pre- and post-infection cellular immune profiles, providing a total of ten trial participants.
Cell-mediated antiviral immunity of the participants was investigated by screening αβ and γδ T-cell receptor repertoires by flow cytometry with 27 different TCR chain-specific monoclonal antibodies (MAbs).
γδ T cells were then ex vivo expanded with 5 mM zoledronic acid, 500 IU/ml IL-2 and

20 ng/ml IL-15.
In the presence and absence of 100 nM recombinant SARS-CoV-2 spike (S) protein to investigate their expansion rate, purity and memory subsets by nucleocounter NC-200 and expression of CD3, TCR/γδ, Vδ2, CD45RA and CD27 by flow cytometry analysis.

### Isolation of PBMC

1. Blood (7.5-8.0 ml) was drawn into a BD Vacutainer CPT Cell Preparation Tube with Sodium Heparin. The tube contains a sodium heparin anticoagulant and a Ficoll-Hypaque density fluid, plus a polyester gel barrier, which separates the two liquids. Centrifuge tube/blood sample at room temperature (18° C. to 25° C.) in a horizontal rotor (swing-out head) for 20 min at 1800×g. Switch centrifuge brakes off.
2. After centrifugation, the sequence of layers occurs as follows (seen from top to bottom): a) plasma—b) peripheral blood mononuclear cells (PBMC) and platelets—c) density solution—d) polyester gel—e) granulocytes—f) red blood cells.
3. Collect a fraction of the plasma layer, leaving 5 to 10 mm of plasma above the interphase without disturbing the cell layer. The plasma can be used for the culture.
4. Harvest the enriched fraction (PBMC) at the interphase with a pipette and transfer to a 15 ml conical tube.
5. Wash the PBMC with 10 ml of phosphate-buffered saline (PBS), by inverting the tube 5 times, and then centrifuge for 5 min at 400×g.
6. Repeat washing steps twice, and then resuspend the cell pellet in 5 ml of PBS. Determine the cell number. Usually 1.3×10⁶ cells are recovered from 1 ml of whole blood.
7. Determine the frequency and phenotype of γδ T cells in PBMC by flow cytometry.
   Ex Vivo Expansion of γδ T cells
8. Centrifuge cell suspensions in 15 ml conical tubes for 5 min at 400×g at room temperature and discard the supernatants.
9. Prepare culture medium (CM) by adding human IL-2 (IL-2), IL-15, and zoledronate (Zometa) to final concentrations of 1000 IU/ml and 5 μM, respectively. ALyS203 (Cell Science & Technology Institute) or OpTmizer (Invitrogen) media support good expansion of γδ T cells. Zometa is provided in liquid form (4 mg/5-ml vial). To prepare a 5 μM solution, add 50 μl of Zometa to 30 ml of culture medium. The culture medium also contains 100 nM recombinant SARS-CoV-2 spike (S) protein.
10. Resuspend cell pellet in culture medium and adjust to 1×10⁶ cells/ml.
11. Pipet 1 ml of CM containing 1×10⁶ cells into each well of a 24-well plate. For large-scale cultures, cells can be seeded at 0.5×10⁶ cells/cm² according to the surface areas of plate wells, dish, or flask.
12. Add autologous plasma, pooled human AB sera, or FCS so that it is approximately 10% of the volume of the culture (100 μl for each well of a 24-well plate). Place the plates in a humidified 37° C., 5% CO₂ incubator for 24-48 hr.
13. Maintain the culture at a cell density of 0.5-2×10⁶ cells/ml. Add fresh medium containing human IL-2 (1000 IU/ml), 20 ng/ml of IL-15, and 100 nM recombinant SARS-CoV-2 spike (S) protein only (without Zometa) every 2-3 days and transfer cultured cells into new wells or flasks as necessary, according to the degree of cell proliferation. Supply plasma or serum to the medium so that the serum concentration can be maintained at least 1%.
14. Harvest cells on day 12-14 and determine the frequency, phenotype, and functions of γδ T cells by flow cytometry.

### Phenotypic Analysis by Flow Cytometry

15. Transfer 200 μl samples containing 2×105 cells to fluorescence-activated cell sorting (FACS) tubes.
16. Add 2 ml of cold PBS and centrifuge for 5 min at 400×g. Then, resuspend the pellets in 50 μl FACS buffer (PBS+1% FCS+0.1% sodium azide). Add 5 μl of each antibodies to the samples.
17. Incubate on ice in the dark for 20 min.
18. Add 2 ml FACS buffer to each sample, and then vortex. Centrifuge samples for 5 min at 400×g at 4° C. Carefully decant the supernatant.
19. Resuspend the cells in 300 μl FACS buffer and vortex. Analyze samples on a flow cytometer.

### IFN-γ Production Assay

20. The day before assay, prepare stimulator cells by culturing 3-5×105 Daudi cells/ml in RPMI 1640 medium plus 10% FCS (RPMI-10) overnight with Zometa (5 PM) (hereafter designated Z-Daudi).
21. Collect Z-Daudi and resuspend in RPMI-10 at 2×10⁶ cells/ml. Add 100 μl of Z-Daudi (2×105) to each well of a round-bottom 96-well plate.
22. Prepare γδ T cells at 2×10⁶ cells/ml in RPMI-10 containing Brefeldin A at 20 μg/ml. Transfer 100 μl of γδ T cell suspension (2×105) to each well containing Z-Daudi cells or to control wells (100 μl of RPMI-10 only, or RPMI-10 with 20 ng/ml of phorbol 12-myristate 13-acetate [PMA] plus 2 μg/ml of ionomycin).
23. Mix by pipetting up and down several times. Incubate for 4 hr in a 37° C., 5% C02 incubator.
24. Centrifuge the plate for 5 min at 400×g at 4° C. and resuspend the pellets in 200 μl of cold PBS.
25. Transfer the samples to FACS tubes. Add 4 ml of cold PBS and centrifuge the tubes for 5 min at 400×g at 4° C.
26. Resuspend the pellets in 50 μl of FACS buffer with FITC-conjugated anti-TCRVγ9 (5 l) and PE/Cy5-conjugated anti-CD3 mAb (2.5 μl). Incubate protected from light for 15 min at room temperature.
27. Add 100 μl of IntraPrep reagent 1 and incubate for 15 min at room temperature. Add 4 ml of PBS to each tube and centrifuge for 5 min at 400×g at room temperature.
28. Remove the supernatant by aspiration and add 100 μl of IntraPrep reagent 2. Incubate for 5 min at room temperature without shaking.
29. Add 5 μl of PE-conjugated anti-IFN-γ mAb to the test tube. Incubate protected from light for 15 min at room temperature.
30. Add 4 ml of PBS to each tube and centrifuge for 5 min at 400×g at room temperature. Remove the supernatant by aspiration and resuspend the cell pellet in 0.5 ml of FACS buffer.
31. Analyze the cells by flow cytometer. Gate on CD3+ TCRVγ9+ cells and examine the expression of IFN-γ.
   Characterization of γδ T cells from Recovered and Seronegative Participants

Plasma and PBMC were analyzed from ten trial participants with confirmed COVID-19 infection within the past 90 days and seven seronegative individuals. Cell-mediated immune responses were evaluated by screening all PBMC subsets by flow cytometry, including αβ and γδ T-cell receptor (TCR) repertoires using 24 TCR Vβ and 3 TCR Vδ chain-specific monoclonal antibodies (MAbs). Pre-infection PBMCs from 3 out of 10 trial subjects were also compared to their respective post-infection samples by immunophenotyping via flow cytometry. Pre- and post-infection γδ T cells were expanded ex vivo in the presence or absence of 100 nM recombinant SARS-CoV-2 spike (S) protein and evaluated for expansion rate, purity and CD45RA⁻CD27⁻ effector memory (EM) and CD45RA⁻CD27⁺ central memory (CM) subset percentages. On day three of expansion, supernatants were analyzed by a flow-based multiplex assay to quantify IL-2, IL-4, IL-10, IL-6, IL-17A, TNF-α, sFas, sFasL, IFN-γ, granzyme A, granzyme B, perforin and granulysin.

### Results

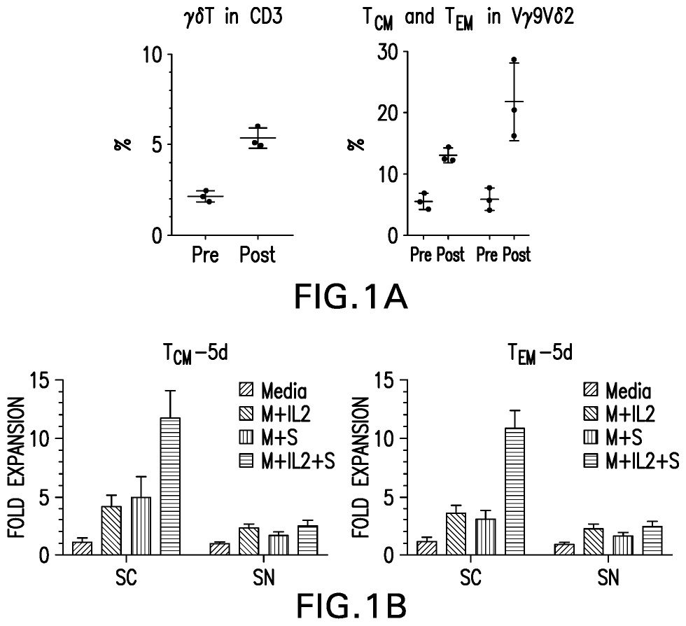

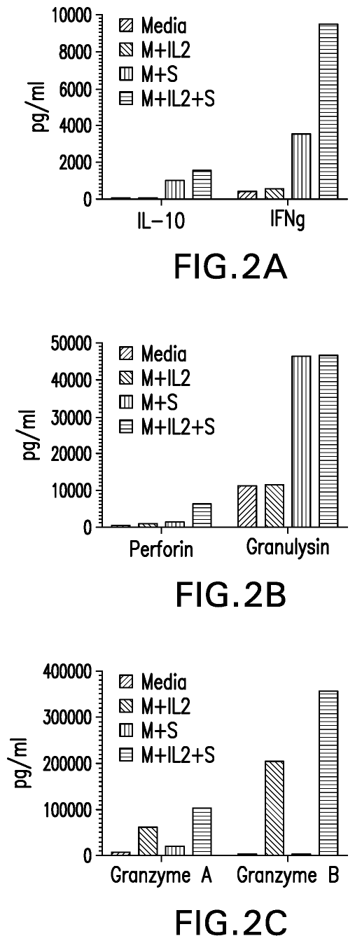

Analyses of the immunophenotyping and T cell repertoires in all ten convalescent patients revealed that γδ T cell populations were activated during the infection. The comparison between pre- and post-infection immune cell profiles in several subjects demonstrated that certain memory subsets of Vγ9Vδ2 T cell populations were also selectively expanded (250%, 975%, and 617% increase in γδ T, Vδ2 EM, and Vδ2 CM cells respectively) (FIG. 1A). There was no change in TCR Vβ chain repertoire post-infection. Ex vivo expansion of pre- and post-infection γδ T cells in the presence or absence of S protein revealed substantial expansion of EM and CM γδ T cells in the post-infection PBMC cultures supplemented with S protein compared to other groups (FIG. 1B). Multiplex cytokine analyses showed increases in IFN-γ, IL-10, granzyme A, granzyme B, perforin and granulysin in γδ T cell cultures supplemented with S protein compared to other groups (FIGS. 2A-C).

Substantial increases in γδ T cell populations were observed in post-COVID19 PBMCs compared to pre-COVID19 and seronegative samples. These cells expanded robustly in the presence of S protein and demonstrated memory cell properties, suggesting an important role of γδ T cells in sustained immunity against SARS-CoV-2. These data suggest potential therapeutic and preventative applications of SARS-CoV-2-specific γδ T cells.

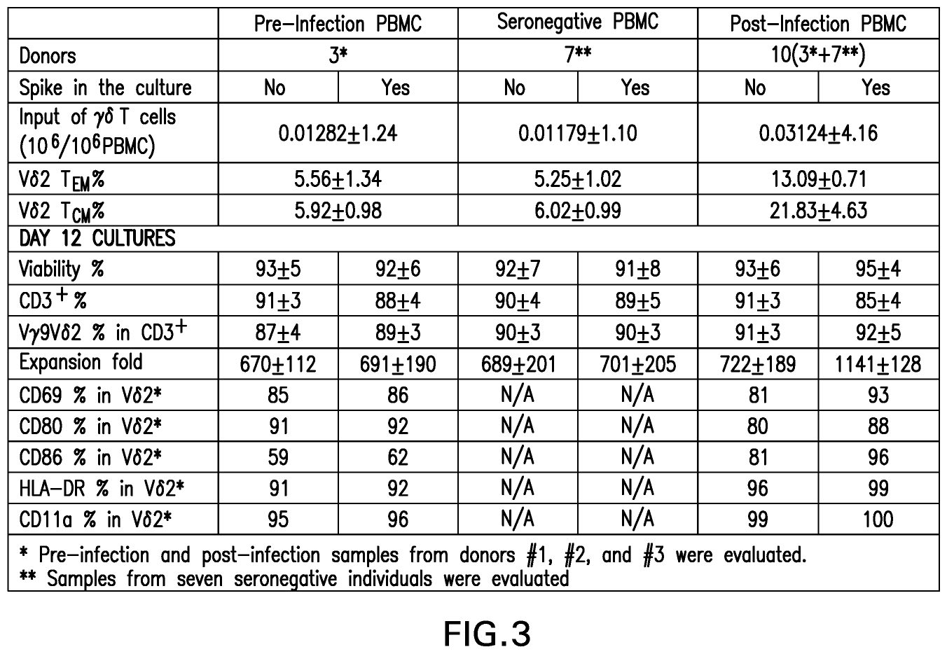

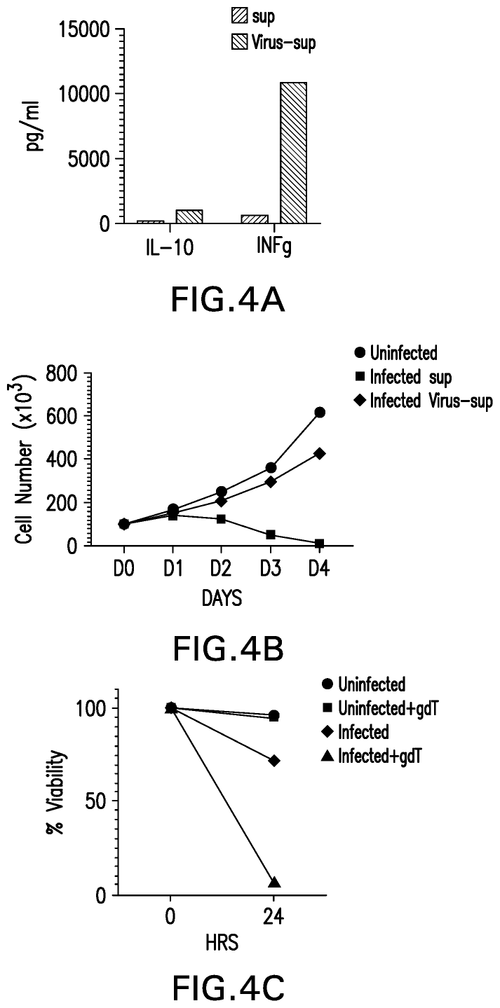

### Example 2 (FIG. 3 and FIG. 4A-C)

### Evaluating Therapeutic and Protective Immunotherapy of SARS-CoV-2-Specific Gamma-Delta T Cells

γδ T cells were expanded ex vivo from pre- and post-infection PBMCs in the presence or absence of 100 nM recombinant SARS-CoV-2 spike (S) protein. To assess their non-cytolytic antiviral activities, expanded cells were exposed to SARS-CoV-2-infected Vero cell culture supernatants. On day 4 after the exposure, γδ T cell supernatants were added to Vero cell cultures 24 h before infecting them with SARS-CoV-2 (MOI 0.01). Cytopathic effects (CPE) of the virus were measured by daily cell count and imaging. Expanded γδ T cells were co-cultured 1:1 with infected (MOI 1) and uninfected ACE2-HEK cells to test their cytolytic antiviral activity. After 24 h, target cell viability was measured by cell counter and FACS. Co-culture supernatants were analyzed by a flow-based multiplex cytokine assay. Expanded γδ T cells were also assessed for markers for activation by CD25 and CD69, for cell exhaustion by CD57, and for antigen presenting cell (APC) functions by CD80, CD86, HLA-DR, CD11a, CXCR5, and CCR7 with flow cytometry. CD45RA⁻CD27⁻ effector memory (EM) and CD45RA⁻CD27⁺ central memory (CM) subsets of expanded γδ T cells were enriched by immunomagnetic separation and co-cultured with B cells harvested from seronegative individuals for assessment of their vaccine effects in vitro. In vitro vaccinated B cells were evaluated for virus-specific activation and the supernatants were collected for antibody titer quantification and virus neutralization assays.

### Results

Expansion rate of γδ T cells, and their EM and CM memory subsets were substantially higher in post-infection PBMCs compared to pre-infection and seronegative controls. Vγ9Vδ2 T cell populations were selectively expanded in ex vivo cultures supplemented with S protein compared to other groups. Moreover, memory subsets of ex vivo expanded SARS-CoV-2-specific γδ T cells displayed potent APC functions, suggesting not only therapeutic, but also potential protective immunotherapy implications of these cells (FIG. 3). Virus-stimulated γδ T cell culture supernatants exhibited increased IFN-γ, IL-10 μlevels and prevented CPE of virus at in vitro viral challenge (FIG. 4A-4B). Isolated expanded γδ T cells effectively killed infected cells in vitro (FIG. 4C). In vitro B cell vaccination results can be determined.

SARS-CoV-2-specific γδ T cells harvested from convalescent individuals exhibited strong non-cytolytic and cytolytic antiviral activities, as well as substantial ex vivo expansion capabilities. Moreover, memory subsets of these cells displayed enhanced APC functions. These data support clinical use of these cells for treatment and prophylaxis of COVID-19 as a cell-based immunotherapy and also as a vaccine candidate generated and expanded ex vivo from SARS-CoV-2-specific convalescent gamma delta T cells. These expanded and enriched compositions of Vγ9Vδ2 T cells are undergoing development and in vivo testing.

### γδT Cells can Induce Both Cellular and Humoral Adaptive Immunity

Markers tested for typing the γδT cells. These include memory, activation, exhaustion and APC markers. Typically, the enriched γδT cells that will be used for the therapeutic and prophylactic compositions described herein will be Vδ2⁺ and CD45RA− in order to be memory T cells. Additionally, the enriched gamma delta cells with have at least two out of three of the following markers: CD11a, HLA-DR, CD86, and will typically express one, two, or all of the following: CCR7, CXCR5, CD69.

Markers tested for typing the γδT cells.

| | |
|---|---|
| | CD3 |
| CD4 | CD8 |
| TCR Vgamma9 | TCR Vdelta1 |
| TCR Vdelta2 | TCR Vdelta3 |
| TCR αβ | TCR γδ |
| CD45RA | CD45RO |
| CD27 | CD56 |
| CD57 | CD25 |
| CD69 | CD62L |
| NKG2D | CD40 |
| CD80 | CD86 |
| CD11b | HLA-DR |
| CCR7 | CD54 |

Additionally, clinical use of these enriched Vγ9Vδ2 T cells (enriched in particular for CD45RA⁻CD27⁻ effector memory (EM) and CD45RA⁻CD27⁺ central memory (CM) cells) for treatment of COVID-19 are expected to shorten the course of the disease, and/or prevent its progression to a more severe form of disease (i.e., avoiding the need for intubation or a ventilator). Shortening the course of COVID-19 can include improving any one or combination of the biomarkers listed in FIG. 5 and improving the overall outcomes of patients with COVID-19, typically when they are early in the course of their disease.

All references cited herein are incorporated by reference to the same extent as if each individual publication, database entry (e.g. Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

1. A composition comprising gamma delta T cells (y5 T cells) which is enriched for Vgamma9 Vdelta2 (Vy9V52) T cells.

2. The composition of claim 1, wherein the composition comprises at least 80% Vgamma9 Vdelta2 (Vy9V52) T cells, and wherein the Vgamma9 Vdelta2 (Vy9V52) cells are specifically enriched for CD45RA“CD27“effector memory (EM) and CD45RA“C-D27⁺ central memory (CM) cells, when compared to a reference or control gamma delta T cell composition.

3. The composition of claim 1, wherein the composition comprises at least 85% Vgamma9 Vdelta2 (Vy9V52) T cells.

4. The composition of claim 1, wherein the composition is capable of eliciting an immune response against a coronavirus when administered to a host.

5. The composition of claim 1, wherein the immune response comprises at least one of a humoral and cellular immune response.

6. A composition enriched for Vgamma9 Vdelta2 (Vγ9V52) T cells produced by a method comprising:
e) isolating y6 T cells from a sample;
f) culturing the y6 T cells with at least one cytokine and a bisphosphonate or bisphosphonate derivative in a suitable medium;
g) further incubating the y6 T cells with a peptide antigen that exhibits at least 70% homology to a coronavirus antigen; and
h) isolating and purifying Vgamma9 Vdelta2 (Vy9V52) T cells to produce the enriched gamma delta T cell composition, wherein the Vgamma9 Vdelta2 (Vy9V52) T cells are specifically enriched for CD45RA“CD27“effector memory (EM) and CD45RA“CD27⁺ central memory (CM) cells, when compared to a reference or control gamma delta T cell composition.

7. The composition of claim 6 wherein the sample is whole blood or PBMCs.

8. The composition of claim 6, wherein the peptide antigen has 80% homology to a coronavirus spike protein.

9. The composition of claim 6, wherein the spike protein sequence shares at least 90% homology with a coronavirus spike protein.

10. The composition of claim 9, wherein the spike protein sequence is SEQ ID NO: 1, or an optimized sequence thereof.

11. The composition of claim 10, wherein, the spike protein sequence can comprise the entire sequence SEQ ID NO: 1 or just the ectodomain amino acids 990-1749 of SEQ ID NO: 1.

12. The composition of claim 6, wherein the purified y5T cells express at least two of the following markers: CD11a, HLA-DR, CD86, CCR7, CXCR5, CD69, and are also CD45RA−.

13. The composition of claim 1, wherein enriched y5T cells exhibit a decrease in two or more T cell exhaustion markers selected from group consisting of CTLA-4, LAG3, BTLA4, and TIM3.

14. The composition of claim 1, wherein the enriched Vgamma9 Vdelta2 (Vy9V52) T cells are allogeneic.

15. The composition of claim 1, wherein the composition comprises at least about 10⁹ purified y6 T cells, wherein the y6 T cells comprise at least about 80% Vgamma9 Vdelta2 positive cells, and wherein the Vgamma9 Vdelta2 (Vy9V52) T cells are specifically enriched for CD45RA“CD27“effector memory (EM) and CD45RA“CD27⁺ central memory (CM) cells, when compared to a reference or control gamma delta T cell composition.

16. A pharmaceutical composition comprising the y5T cells of claim 1.

17. The pharmaceutical composition of claim 16, wherein the composition comprises, optionally, a pharmaceutically acceptable carrier, diluent, adjuvant and/or additive.

18. The composition of claim 1, which is capable of inducing an immunological response against coronavirus in a subject.

19. The composition of claim 18, wherein the method includes inducing a humoral and cellular response against coronavirus.

20. The composition of claim 19, wherein the immune response is induced by a regimen comprising one or at least two administrations.

21. The composition of claim 6, wherein the coronavirus is SAR-S-CoV-2 (COVID-19).

22. The composition of claim 1, for use in diminishing or preventing a coronavirus infection in a mammalian subject.

23. The composition of claim 22, wherein said diminishing or preventing comprises inducing coronavirus-specific immunity against SARS-CoV-2 (COVID-19).

24. A method of diminishing or preventing a coronavirus infection in a mammalian subject comprising administering an effective amount of the y5T cells or compositions of claim 1 to the subject.

25. A method of inducing cellular and or humoral immunity in a mammalian subject, comprising administering an effective amount of the y5T cells or compositions of claim 1 to the subject.

26. A method of eliciting an immune response in a subject, comprising administering an effective amount of the y5T cells or compositions of claim 1 to the subject.

27. A method of inducing neutralizing antibodies against SARS-CoV-2 in a subject, comprising administering an effective amount of the y5T cells or compositions of claim 1 to the subject.

28. The method of claim 24, wherein the method includes inducing a humoral response against the coronavirus.

29. The method of claim 28, wherein the humoral response is induced by a regimen comprising at least one or at least two administrations.

30. The method of claim 24, further comprising administering an effective amount of at least one second therapeutic agent selected from the group consisting of: an antiviral agent, antibacterial agent, an angiotensin receptor blocker (ARB), an IL-6 inhibitor, hydroxychloroquine, chloroquine, an anticoagulant or and COVID-19 immune serum or plasma.

31. The method of claim 30, wherein the at least one second therapeutic agent is an antiviral agent.

32. The method of claim 31, wherein the antiviral agent is favipiravir.

33. The method of claim 31, wherein the antiviral agent is remdesivir.

34. The method of claim 30, wherein the at least one second therapeutic agent is an antibacterial agent.

35. The method of claim 34, wherein the antibacterial agent is selected from the group consisting of azithromycin, tobramycin, aztreonam, ciprofloxacin, meropenem, cefepime, cetadizine, imipenem, piperacillin-tazobactam, amikacin, gentamicin and levofloxacin.

36. The method of claim 35, wherein the antibacterial agent is azithromycin.

37. The method of claim 30, wherein the at least one second therapeutic agent is an ARB.

38. The method of claim 37, wherein the ARB is losartan.

39. The method of claim 37, wherein the ARB is valsartan.

40. The method of claim 30, wherein the at least one second therapeutic agent is an IL-6 inhibitor.

41. The method of claim 40, wherein the IL-6 inhibitor is selected from the group consisting of: an anti-IL-6 receptor antibody or an antigen binding fragment thereof, an anti-IL-6 antibody or an antigen binding fragment thereof, and a JAK/STAT inhibitor.

42. The method of claim 41, wherein the IL-6 inhibitor is an anti-IL-6 receptor antibody, or antigen binding fragment thereof.

43. The method of claim 42, wherein the anti-IL-6 receptor antibody is tocilizumab or sarilumab.

44. The method of claim 41, wherein IL-6 inhibitor is an anti-IL-6 antibody, or antigen binding fragment thereof.

45. The method of claim 44, wherein the anti-IL-6 antibody is selected from the group consisting of ziltivekimab, siltuximab, gerilimzumab, sirukumab, clazakizumab, olokizumab, VX30 (VOP-R003; Vaccinex), EB-007 (EBL029; Eleven Bio), and FM101 (Femta Pharmaceuticals, Lonza).

46. The method of claim 41, wherein the IL-6 inhibitor is a JAK/STAT inhibitor.

47. The method of claim 46, wherein the JAK/STAT inhibitor is selected from the group consisting of ruxolotinib, tofacitinib, and baricitinib.

48. The method of claim 30, wherein the at least second one second therapeutic agent is an anticoagulant.

49. The method of claim 48, wherein the anticoagulant is selected from the group consisting of enoxaparin, heparin, low-molecular weight heparin, dabigatran, rivaroxaban, apixaban, edoxaban, and fondaparinux.

50. A kit comprising a container comprising the composition of enriched gamma delta T cells of claim 1, and instructions for using the kit.

51. The kit of claim 50, wherein the kit further comprises a separate container comprising normal saline.

52. The kit of claim 50, wherein the kit further comprises materials suitable for intravenous administration and optionally additional materials for oral administration.


SEQUENCE LISTING (1 sequence)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT